(19) United States
(12) Patent Application Publication
(10) Pub. No.: US 2026/0253126 A1
(43) Pub. Date: Aug. 27, 2026

(54) SYSTEM

(71) Applicant: SoftBank Group Corp., Tokyo (JP)

(72) Inventor: Taiga TADA, Tokyo (JP)

(73) Assignee: SoftBank Group Corp., Tokyo (JP)

(21) Appl. No.: 19/537,544

(22) Filed: Feb. 12, 2026

(30) Foreign Application Priority Data
Feb. 21, 2025 (JP) 2025-027023

(51) Int. Cl. G06Q 30/0601; G06Q 10/083; G16H 20/60

(52) U.S. Cl. CPC G06Q 30/06313; G06Q 10/0843; G06Q 30/0633; G16H 20/60

(57) ABSTRACT

The system according to the embodiment comprises an acquisition unit, a generation unit, a list generation unit, and a suggestion unit. The acquisition unit acquires health data. The generation unit analyzes the data acquired by the acquisition unit and generates a meal plan according to the user's health condition, goals, preferences, and food allergies. The list generation unit automatically generates a shopping list based on the meal plan generated by the generation unit. The suggestion unit makes suggestions based on the shopping list generated by the list generation unit.

### CROSS-REFERENCE TO RELATED APPLICATION

The present application claims priority to and incorporates by reference the entire contents of Japanese Patent Application No. 2025-027023 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there is room for improvement because meal plans tailored to a user's health condition, goals, preferences, and food allergies have not been efficiently generated, and shopping lists have not been automatically generated sufficiently.

### SUMMARY OF THE INVENTION

The system according to the embodiment comprises an acquisition unit, a generation unit, a list generation unit, and a suggestion unit. The acquisition unit acquires health data. The generation unit analyzes the data acquired by the acquisition unit and generates a meal plan according to the user's health condition, goals, preferences, and food allergies. The list generation unit automatically generates a shopping list based on the meal plan generated by the generation unit. The suggestion unit makes suggestions based on the shopping list generated by the list generation unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

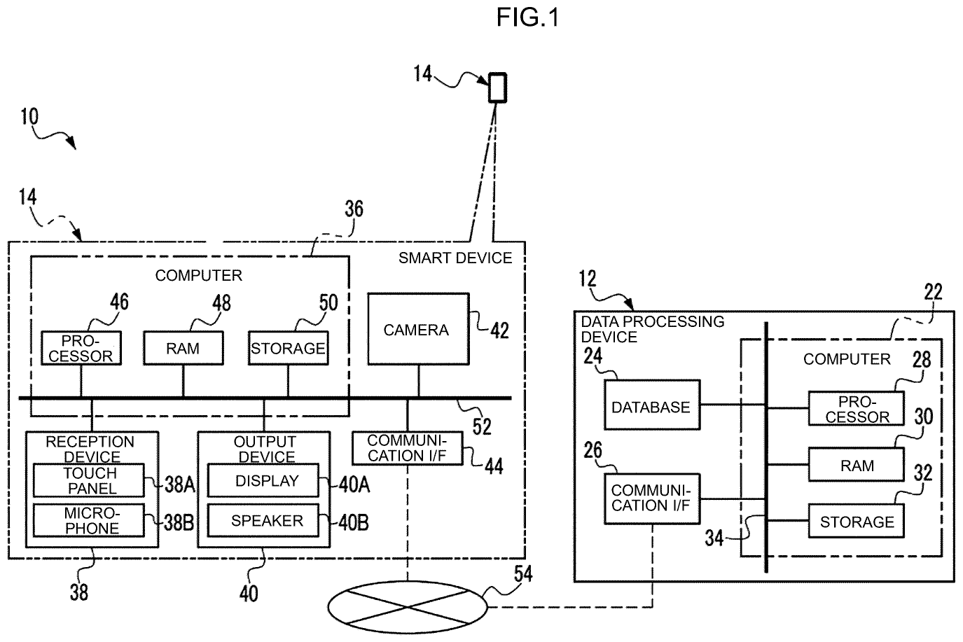

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

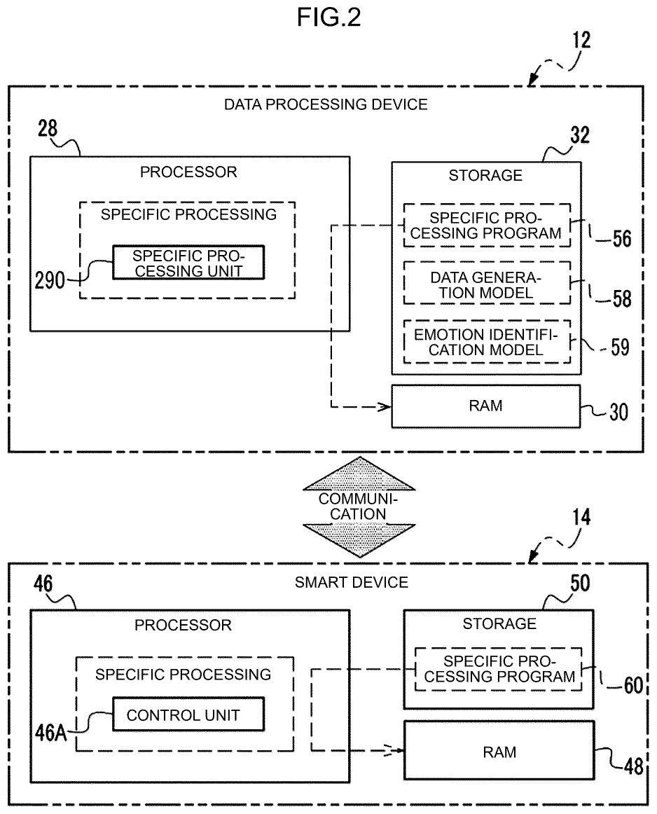

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system that connects to health devices and automatically generates a meal plan according to a user's health condition, goals, preferences, and food allergies. This system acquires the user's health data from health devices, and AI analyzes the data to generate a meal plan tailored to the user's health condition, goals, preferences, and food allergies. The generated meal plan includes a menu that takes into account quantities that prevent food waste, and a shopping list is also automatically generated. Furthermore, optimal suggestions are made according to the user's shopping situation. For example, when the user is able to go shopping, the system suggests the optimal supermarket based on online flyers. If the user does not have time to go shopping, ingredients are purchased from an online supermarket and arranged to arrive when the user is about to start cooking. If the user does not have time to cook, food delivery in accordance with the meal plan is suggested. Thus, the system connected to health devices can automatically generate a meal plan according to the user's health condition, goals, preferences, and food allergies, and reduce the burden of shopping and cooking. Specifically, the system automatically acquires various health data such as heart rate (one-dimensional time series array, e.g., minute-by-minute heart rate data), blood pressure (two-dimensional array, e.g., date×measurement value), body weight (one-dimensional array, e.g., daily body weight values), and sleep patterns (time series array, e.g., sleep start/end times, depth labels) from health devices such as wearable devices, scales, blood pressure monitors, and sleep trackers via Bluetooth or Wi-Fi communication. The system performs preprocessing such as noise removal, normalization, and missing value imputation on these data in a preprocessing unit before inputting them to an AI analysis unit. The AI analysis unit uses, for example, multilayer perceptrons, convolutional neural networks (CNN), recurrent neural networks (RNN) for time series analysis, or Transformer-based models to estimate the user's health condition (e.g., obesity score, blood pressure risk label, sleep quality score) from the health data. Examples of AI input include (1) one week of heart rate time series data (168-dimensional vector), (2) past 30 days of body weight trends (30-dimensional vector), and (3) time series array of sleep depth (7 days×8 hours×1 dimension). Examples of AI output include (1) health condition labels (e.g., normal, caution, needs improvement), (2) goal achievement score (continuous value from 0.0 to 1.0), (3) recommended calorie intake (numeric value), and (4) allergy risk assessment (binary label). These outputs are passed to the subsequent meal plan generation unit, which generates a meal plan optimized for the user's health condition and goals (e.g., 1800 kcal per day, 80 g protein, excluding nuts, Japanese cuisine-focused). The meal plan generation unit collaborates with ingredient and recipe databases and applies algorithms (e.g., integer programming, graph search algorithms) to optimize ingredient usage plans on a weekly and daily basis to minimize food waste. The shopping list generation unit automatically extracts the necessary ingredients and quantities from the generated menu, integrates duplicate ingredients, refers to inventory status, and generates a minimal shopping list. The suggestion unit collects the user's current location information, schedule, and online flyer information (JSON format price, store, discount information), and recommends the optimal supermarket using multivariate scoring based on price, distance, assortment, etc. If the user cannot go shopping, the system collaborates with online supermarket APIs to automatically place orders and arrange delivery, considering available delivery times, inventory status, and delivery fees. If the user does not have time to cook, the system collaborates with food delivery service APIs to automatically search for and suggest menus that match the meal plan (e.g., low-calorie bento, allergy-friendly menus). This series of processes is realized by advanced application of computer technologies such as high-dimensional data analysis, multi-module collaboration, and real-time optimization, which differ from conventional manual work or simple rule-based processing, thereby providing technical effects such as significant improvement in processing speed, increased suggestion accuracy, reduction of food waste, and reduction of user burden. Specific application fields include personal health management support, lifestyle disease prevention, corporate welfare services, meal management in nursing care facilities, and nutrition management for athletes.

The system according to the embodiment comprises an acquisition unit, a generation unit, a list generation unit, and a suggestion unit. The acquisition unit acquires health data. Health data may include, for example, heart rate, blood pressure, body weight, and sleep patterns, but is not limited to these examples. The acquisition unit may, for example, use a wearable device to measure heart rate and collect data. The acquisition unit may also use a blood pressure monitor to measure blood pressure and collect data. Furthermore, the acquisition unit may use a scale to measure body weight and collect data. For example, the acquisition unit may use a sleep tracker to measure sleep patterns and collect data. The generation unit analyzes the data acquired by the acquisition unit and generates a meal plan according to the user's health condition, goals, preferences, and food allergies. The generation unit may, for example, use AI to analyze the data and evaluate the user's health condition. Based on the user's goals, the generation unit may generate, for example, a low-calorie and nutritionally balanced meal plan if the user aims for dieting. The generation unit may also generate a meal plan including preferred ingredients based on the user's preferences. Furthermore, the generation unit may generate a meal plan excluding nuts if the user has a nut allergy, based on the user's food allergies. The list generation unit automatically generates a shopping list based on the meal plan generated by the generation unit. The list generation unit may, for example, list the types and quantities of necessary ingredients. The list generation unit enables the user to efficiently purchase necessary ingredients when shopping. The suggestion unit makes optimal suggestions based on the shopping list generated by the list generation unit. The suggestion unit may, for example, suggest the optimal supermarket based on online flyers when the user is able to go shopping. The suggestion unit may arrange for ingredients to be purchased from an online supermarket and delivered to arrive when the user is about to start cooking if the user does not have time to go shopping. The suggestion unit may suggest food delivery in accordance with the meal plan if the user does not have time to cook. Thus, the system according to the embodiment can automatically generate a meal plan according to the user's health condition, goals, preferences, and food allergies, automatically generate a shopping list, and make optimal suggestions. Specifically, the system automatically acquires various health data such as heart rate (minute-by-minute time series array, e.g., 168-dimensional vector for one week), blood pressure (two-dimensional array, e.g., date×measurement value), body weight (one-dimensional array, e.g., daily body weight values), and sleep patterns (time series array, e.g., sleep start/end times, depth labels) from wearable devices, scales, blood pressure monitors, and sleep trackers equipped with Bluetooth or Wi-Fi communication functions as the acquisition unit. The acquisition unit passes these data to the preprocessing unit, which performs preprocessing such as noise removal (e.g., moving average filter), normalization (e.g., Z-score normalization), and missing value imputation (e.g., linear interpolation). The generation unit inputs the preprocessed data to the AI analysis unit, which uses neural networks such as multilayer perceptrons, convolutional neural networks (CNN), recurrent neural networks (RNN), and Transformer-based models to estimate health condition. Examples of AI input include (1) one week of heart rate time series data (168-dimensional vector), (2) past 30 days of body weight trends (30-dimensional vector), and (3) time series array of sleep depth (7 days×8 hours×1 dimension). Examples of AI output include (1) health condition labels (e.g., normal, caution, needs improvement), (2) goal achievement score (continuous value from 0.0 to 1.0), (3) recommended calorie intake (numeric value), and (4) allergy risk assessment (binary label). These outputs are passed to the subsequent meal plan generation unit, which generates a meal plan optimized for the user's health condition and goals (e.g., 1800 kcal per day, 80 g protein, excluding nuts, Japanese cuisine-focused). The meal plan generation unit collaborates with ingredient and recipe databases and applies algorithms (e.g., integer programming, graph search algorithms) to optimize ingredient usage plans on a weekly and daily basis to minimize food waste. The list generation unit automatically extracts the necessary ingredients and quantities from the generated menu, integrates duplicate ingredients, refers to inventory status, and generates a minimal shopping list. The suggestion unit collects the user's current location information, schedule, and online flyer information (JSON format price, store, discount information), and recommends the optimal supermarket using multivariate scoring based on price, distance, assortment, etc. If the user cannot go shopping, the system collaborates with online supermarket APIs to automatically place orders and arrange delivery, considering available delivery times, inventory status, and delivery fees. If the user does not have time to cook, the system collaborates with food delivery service APIs to automatically search for and suggest menus that match the meal plan (e.g., low-calorie bento, allergy-friendly menus). This series of processes is realized by advanced application of computer technologies such as high-dimensional data analysis, multi-module collaboration, and real-time optimization, which differ from conventional manual work or simple rule-based processing, thereby providing technical effects such as significant improvement in processing speed, increased suggestion accuracy, reduction of food waste, and reduction of user burden. Specific application fields include personal health management support, lifestyle disease prevention, corporate welfare services, meal management in nursing care facilities, and nutrition management for athletes.

The suggestion unit can suggest a supermarket based on online flyers when the user is able to go shopping. For example, when the user is able to go shopping, the suggestion unit suggests the optimal supermarket based on online flyers. Online flyers may include flyers or discount information for specific supermarkets, but are not limited to these examples. The suggestion unit may select the optimal supermarket based on criteria such as price, distance, and assortment. By suggesting the optimal supermarket when the user is able to go shopping, efficient shopping becomes possible. Specifically, the suggestion unit is equipped with a web scraping module and API collaboration module for automatically collecting flyer information published online in formats such as JSON or CSV. The suggestion unit extracts structured data from the collected flyer data, such as price information for each supermarket (e.g., product ID, price, discount rate), store location information (latitude and longitude), business hours, and assortment lists (product categories, inventory status). The suggestion unit receives as input the user's current location information (GPS coordinates), schedule information (calendar API collaboration), and the necessary ingredient list (e.g., ingredient ID, required quantity) received from the shopping list generation unit. The suggestion unit inputs these data into a multivariate scoring algorithm (e.g., weighted sum score, TOPSIS method, ranking by random forest) and calculates a “comprehensive convenience score” for each supermarket. For example, input examples include (1) user's current location (latitude 35.6, longitude 139.7), (2) necessary ingredient list (e.g., 2 packs of eggs, 500 g chicken, 1 bunch of spinach), (3) price, distance, and assortment information for Supermarket A (e.g., eggs 198 yen, chicken in stock, distance 1.2 km), (4) information for Supermarket B (eggs 210 yen, chicken out of stock, distance 0.8 km), etc. The suggestion unit considers user setting parameters such as “price priority,” “distance priority,” and “assortment priority” and outputs the optimal supermarket in ranking format. Output examples include (1) recommended supermarket name (e.g., Supermarket A), (2) reason for recommendation (e.g., all necessary ingredients available, lowest total price), (3) estimated required time (e.g., 15 minutes on foot), and (4) discount information (e.g., 20 yen off coupon for eggs valid). The suggestion unit passes these outputs to the user interface unit, which executes subsequent processing such as map display, route guidance, and presentation of discount coupons. Thus, the suggestion unit can perform real-time optimization processing that simultaneously considers multiple quantitative criteria, unlike conventional simple store list presentation or intuitive human selection, thereby greatly improving shopping efficiency and reducing unnecessary movement and costs. Application fields include personal shopping support apps, family health management systems, ingredient procurement optimization for nursing care facilities, and corporate welfare services.

The suggestion unit can arrange for ingredients to be purchased from an online supermarket and delivered to arrive when the user is about to start cooking if the user does not have time to go shopping. For example, when the user does not have time to go shopping, the suggestion unit arranges for ingredients to be purchased from an online supermarket and delivered to arrive when the user is about to start cooking. Online supermarkets may include specific online stores or delivery areas, but are not limited to these examples. The suggestion unit arranges based on criteria such as delivery time settings and order procedures. By purchasing ingredients from an online supermarket when the user does not have time to go shopping, the user can efficiently start cooking. Specifically, the suggestion unit is equipped with a communication module for collaborating with online supermarket APIs and automatically converts the necessary ingredient list (e.g., ingredient ID, required quantity, preferred brand) received from the shopping list generation unit into the cart structure of the online supermarket. The suggestion unit receives as input the user's schedule information (e.g., scheduled return home today between 18:00 and 19:00), delivery address, and past order history, and automates inventory inquiry, acquisition of available delivery time slots, order confirmation, and payment processing with the online supermarket API. Input examples include (1) necessary ingredient list (e.g., 1 L milk, 3 tomatoes, 400 g chicken thigh), (2) user's scheduled return home time (e.g., 19:00), (3) available delivery time slots for Online Supermarket A (e.g., 18:30-20:00), (4) inventory status (e.g., milk in stock, tomatoes in stock, chicken thigh out of stock), etc. The suggestion unit applies delivery time optimization algorithms (e.g., time matching, delivery slot optimization, partial order splitting) based on this information and arranges delivery to match the scheduled cooking start time. Output examples include (1) order confirmation details (e.g., 1 L milk, 3 tomatoes, substitute 400 g pork thigh for chicken thigh), (2) scheduled delivery time (e.g., expected arrival at 18:45), (3) payment amount (e.g., total 1,280 yen), and (4) delivery status tracking URL. After order confirmation, the suggestion unit notifies the user and executes subsequent processing such as real-time tracking of delivery status and updating of arrival predictions. Thus, the suggestion unit automates collaboration with multiple online supermarket APIs, inventory status, and delivery slot optimization, unlike conventional manual ordering or human delivery coordination, thereby realizing ingredient delivery optimized for the user's cooking start timing and greatly reducing time loss and order errors. Application fields include ingredient procurement support for dual-income households, automatic ingredient ordering for nursing care facilities, and ingredient delivery services as corporate welfare.

The suggestion unit can suggest food delivery in accordance with the meal plan when the user does not have time to cook. For example, when the user does not have time to cook, the suggestion unit suggests food delivery in accordance with the meal plan. Food delivery may include specific restaurants or types of cuisine, but is not limited to these examples. The suggestion unit may suggest menus tailored to the user's health condition and goals and arrange for food delivery. By suggesting food delivery in accordance with the meal plan when the user does not have time to cook, the user can easily enjoy healthy meals. Specifically, the suggestion unit is equipped with an integrated API module for collaborating with food delivery service APIs (e.g., multiple food delivery services). The suggestion unit receives as input meal plan information (e.g., 1800 kcal per day, 80 g protein, excluding nuts, Japanese cuisine-focused) and the user's health condition, allergy information, and preferences (e.g., low-calorie, excluding dairy products, no spicy foods) from the generation unit. The suggestion unit applies algorithms for filtering and scoring restaurant and menu information (e.g., menu ID, nutritional content, allergen information, price, available delivery time) obtained from food delivery service APIs, such as rule-based filtering plus neural network-based health score estimation. Input examples include (1) user's health condition (e.g., obesity score 0.7, nut allergy), (2) meal plan requirements (e.g., less than 600 kcal per meal, at least 25 g protein), (3) menu information from Food Delivery Service A (e.g., low-calorie bento, 550 kcal, 28 g protein, no nuts, price 900 yen), etc. The suggestion unit outputs the optimal food delivery menu in ranking format based on multivariate criteria such as health score, allergy compatibility, price, and delivery time. Output examples include (1) recommended menu name (e.g., low-calorie Japanese bento), (2) reason for recommendation (e.g., meets all meal plan requirements, no allergy risk), (3) estimated delivery time (e.g., expected arrival at 19:10), and (4) order link. When the user confirms the order, the suggestion unit executes subsequent processing such as automatic ordering, payment, and delivery status tracking via the food delivery service API. Thus, the suggestion unit can automate optimal food delivery suggestions that simultaneously meet health condition, allergy, and nutritional requirements, unlike conventional human menu selection or simple restaurant list presentation, thereby realizing healthy and highly convenient meal choices. Application fields include food delivery collaboration for health management apps, meal arrangement for nursing and medical facilities, and nutrition management support for athletes.

The generation unit can generate a menu that takes into account quantities that prevent food waste. For example, the generation unit generates a menu that takes into account quantities that prevent food waste. Quantities that prevent food waste may include appropriate quantity calculation methods and storage methods, but are not limited to these examples. The generation unit may, for example, organize menus so that ingredients for one week are used up without waste. By generating a menu that takes into account quantities that prevent food waste, ingredient waste can be reduced and economic benefits can be expected. Specifically, the generation unit collaborates with ingredient databases (e.g., ingredient ID, unit, storage period, minimum sales unit, inventory status) and recipe databases (e.g., recipe ID, required ingredients, quantities, number of cooking days), and applies algorithms (e.g., integer programming, graph search algorithms, dynamic programming) to optimize ingredient usage plans on a weekly and daily basis. The generation unit receives as input the user's meal plan (e.g., three meals per day, one week's menu), inventory ingredient list, minimum purchasable units, and storage periods. Input examples include (1) one week's meal plan (e.g., Monday breakfast: egg sandwich, lunch: chicken salad, dinner: sautéed spinach . . . ), (2) inventory ingredient list (e.g., 10 eggs, 500 g chicken, 2 bunches of spinach), (3) minimum sales unit for ingredients (e.g., one pack of eggs: 10 eggs, one pack of chicken: 500 g), (4) storage period (e.g., eggs: 14 days, chicken: 3 days, spinach: 2 days), etc. The generation unit optimizes the ingredient usage schedule based on this information and adjusts recipe combinations and cooking order to prevent surplus ingredients. Output examples include (1) optimized one-week menu list, (2) ingredient usage plan for each ingredient (e.g., eggs: used on Monday morning, Wednesday lunch, Friday night, all used up), (3) shopping list with zero surplus ingredients, etc. The generation unit passes these outputs to the shopping list generation unit, which executes subsequent processing such as automatic extraction of necessary ingredients and quantities and inventory management. Thus, the generation unit can automate optimization processing that simultaneously considers multiple constraints (storage period, minimum unit, inventory status), unlike conventional manual work or simple recipe selection, thereby providing technical effects such as significant reduction of food waste, reduction of economic burden, and reduction of environmental impact. Application fields include household ingredient management apps, ingredient ordering optimization for nursing care facilities, and inventory management for corporate cafeterias.

The acquisition unit can acquire health data including heart rate, blood pressure, body weight, and sleep patterns. For example, the acquisition unit acquires health data including heart rate, blood pressure, body weight, and sleep patterns. Heart rate may be acquired using a wearable device, blood pressure may be acquired using a blood pressure monitor, body weight may be acquired using a scale, and sleep patterns may be acquired using a sleep tracker. By acquiring health data such as heart rate, blood pressure, body weight, and sleep patterns, the user's health condition can be understood. Specifically, the acquisition unit is equipped with device driver modules and communication protocol conversion modules for automatically acquiring data from wearable devices, scales, blood pressure monitors, and sleep trackers equipped with Bluetooth or Wi-Fi communication functions. The acquisition unit automatically acquires heart rate data (e.g., minute-by-minute time series array, 168-dimensional vector for one week), blood pressure data (e.g., two-dimensional array of date×measurement value), body weight data (e.g., one-dimensional array of daily body weight values), and sleep pattern data (e.g., time series array of sleep start/end times and depth labels). Input examples include (1) heart rate data (e.g., 2024/6/1 08:00 72 bpm, 08:01 74 bpm . . . ), (2) blood pressure data (e.g., 2024/6/1 08:00 120/80 mmHg . . . ), (3) body weight data (e.g., 2024/6/1 65.2 kg . . . ), (4) sleep pattern data (e.g., 2024/5/31 23:00-2024/6/1 06:30, depth: light→deep→light), etc. The acquisition unit passes these data to the preprocessing unit, which performs preprocessing such as noise removal (e.g., moving average filter), normalization (e.g., Z-score normalization), and missing value imputation (e.g., linear interpolation). Thus, the acquisition unit realizes real-time automatic acquisition from multiple devices, high-precision data collection, and data quality standardization, unlike conventional manual work or simple recording, thereby providing technical effects such as improved accuracy of health condition estimation and faster abnormality detection. Application fields include personal health management apps, remote monitoring for medical institutions, and health condition monitoring for nursing care facilities.

The acquisition unit can estimate the user's emotions and adjust the timing of health data acquisition based on the estimated emotions of the user. For example, if the user is feeling stressed, the acquisition unit adjusts to acquire health data during relaxed periods. If the user is tired, the acquisition unit adjusts to acquire health data during rest. If the user is active, the acquisition unit adjusts to acquire health data after exercise. By adjusting the timing of health data acquisition according to the user's emotions, data can be acquired at more appropriate times. Emotion estimation is realized using, for example, emotion engines or generative AI with emotion estimation functions. Generative AI may include text generation AI (e.g., LLM) or multimodal generative AI, but is not limited to these examples. Specifically, the acquisition unit collects various multimodal inputs for emotion estimation, such as voice data (e.g., one minute of speech waveform, 16 kHz sampling, 960,000 samples), facial image data (e.g., 128×128 pixel RGB image), and text data (e.g., SNS post, about 100 tokens). The acquisition unit performs noise removal (e.g., spectral subtraction, face detection/cropping, text normalization) and feature extraction (e.g., MFCC acoustic features, facial expression vectors, BERT embedding vectors) in the preprocessing unit and inputs them to the emotion estimation AI analysis unit. The emotion estimation AI analysis unit uses, for example, multimodal Transformers that handle voice, image, and text integratively, ensemble models combining convolutional neural networks for voice, ResNet for facial images, and large language models for text. Examples of AI input include (1) speech waveform saying “I'm tired today”+facial image+text, (2) smiling facial image+SNS post saying “Fun!”, (3) expressionless facial image+post saying “I'm stressed”, etc. Examples of AI output include (1) emotion labels (e.g., stress, relaxation, fatigue, activity), (2) emotion intensity score (continuous value from 0.0 to 1.0), and (3) emotion change trend (e.g., stress increased over the past 24 hours). Based on these emotion estimation results, the acquisition unit applies health data acquisition timing optimization algorithms (e.g., lower acquisition frequency during stress, higher frequency during relaxation, prioritize heart rate and blood pressure acquisition after activity) and automatically adjusts the timing of data acquisition from health devices via Bluetooth or Wi-Fi communication. Subsequent processing includes recording acquired health data in time series and passing data to the AI analysis unit for analyzing health indicator fluctuations by emotional state. This configuration realizes health data collection optimized for the user's psychological and physiological state by combining high-precision emotion estimation by multimodal AI and real-time acquisition timing optimization, unlike conventional simple scheduled acquisition or subjective human judgment, thereby providing technical effects such as improved data reliability, faster abnormality detection, and reduced user burden. Application fields include stress management apps, mental health support, remote medical monitoring, and health management in nursing care facilities.

The acquisition unit can analyze the user's past health data and select an optimal acquisition method. For example, the acquisition unit selects a method to acquire data during the most stable time periods based on the user's past health data. The acquisition unit may select a method to acquire data during time periods when specific health indicators are likely to fluctuate, based on the user's past health data. The acquisition unit may also select a method to optimize the frequency of data acquisition by analyzing the user's past health data. By analyzing the user's past health data, the optimal acquisition method can be selected. Specifically, the acquisition unit automatically acquires time series health data accumulated for each user (e.g., heart rate minute-by-minute 168-dimensional vector, daily blood pressure two-dimensional array, daily body weight one-dimensional array, sleep pattern 7 days×8 hours array) from the database. The acquisition unit applies time series analysis algorithms (e.g., autoregressive models, moving averages, seasonal decomposition, coefficient of variation calculation) to quantitatively evaluate the stability and variability of each health indicator. Input examples include (1) past 30 days of heart rate time series data, (2) past month of blood pressure trends, (3) past week of sleep depth array, etc. The AI analysis unit outputs (1) stability score (e.g., standard deviation, coefficient of variation), (2) fluctuation peak time (e.g., heart rate increases at 22:00 every day), (3) optimal acquisition frequency (e.g., three times a day, every two hours), etc. Based on these output results, the acquisition unit automatically generates a health data acquisition scheduler and optimizes the timing and frequency of data acquisition from health devices via Bluetooth or Wi-Fi communication. Subsequent processing includes collecting health data in real time according to the optimized acquisition schedule and passing data to the abnormal value detection or health condition estimation AI analysis unit. This configuration realizes data acquisition optimized for each user's lifestyle rhythm and health condition by combining AI-based time series analysis and dynamic scheduling, unlike conventional acquisition based on human experience or fixed schedules, thereby providing technical effects such as improved data utility, increased abnormality detection accuracy, and efficient use of communication and computational resources. Application fields include personal health management apps, remote monitoring for medical institutions, and health condition monitoring for nursing care facilities.

The acquisition unit can perform filtering based on the user's current living conditions and activity level when acquiring health data. For example, if the user is exercising, the acquisition unit acquires only data related to exercise. If the user is resting, the acquisition unit acquires only data related to relaxation. If the user is working, the acquisition unit acquires only data related to stress level. By acquiring data according to the user's living conditions and activity level, more accurate health data can be obtained. Specifically, the acquisition unit acquires living condition and activity level-related data in real time from the user's smartphone or wearable device, such as accelerometer data (e.g., three-axis acceleration, 60 samples per second), location information (e.g., GPS coordinates), and calendar information (e.g., work schedule, break schedule). The acquisition unit performs noise removal and feature extraction in the preprocessing unit (e.g., step count and exercise intensity estimation from acceleration, movement speed calculation from GPS, activity labeling from calendar) and inputs the data to the activity recognition AI analysis unit. The AI analysis unit uses, for example, time series convolutional neural networks or LSTM-based time series classification models to estimate activity labels such as “exercising,” “resting,” or “working” from the input data. Input examples include (1) accelerometer data (e.g., high-intensity variation→exercising), (2) calendar “meeting”+low acceleration→working, (3) no GPS movement+nighttime→resting, etc. AI output examples include (1) activity label (exercising, resting, working), (2) activity intensity score (0.0-1.0), (3) activity duration (e.g., exercising for 30 minutes), etc. Based on these output results, the acquisition unit applies health data acquisition filtering logic (e.g., acquire heart rate and calorie consumption during exercise, acquire sleep depth and relaxation level during rest, acquire stress indicators during work) and selectively acquires only the necessary health data. Subsequent processing includes recording acquired data with activity labels and passing data to the activity-specific health condition estimation or abnormality detection AI analysis unit. This configuration realizes high-precision health data collection with less noise and reduction of unnecessary data by combining AI-based activity recognition and dynamic data filtering, unlike conventional uniform data acquisition or manual selection by humans, thereby providing technical effects such as improved analysis accuracy. Application fields include training management for athletes, health management support for companies, and activity monitoring for nursing care facilities.

The acquisition unit can estimate the user's emotions and determine the priority of health data to be acquired based on the estimated emotions of the user. For example, if the user is feeling stressed, the acquisition unit prioritizes acquisition of data related to stress level. If the user is tired, the acquisition unit prioritizes acquisition of data related to fatigue. If the user is relaxed, the acquisition unit prioritizes acquisition of data related to relaxation. By determining the priority of health data according to the user's emotions, important data can be acquired preferentially. Emotion estimation is realized using, for example, emotion engines or generative AI with emotion estimation functions. Generative AI may include text generation AI (e.g., LLM) or multimodal generative AI, but is not limited to these examples. Specifically, the acquisition unit collects multimodal information such as voice, facial image, and text data from the user, performs feature extraction in the preprocessing unit (e.g., MFCC from voice, facial expression features from facial images, emotion word extraction from text), and inputs the data to the emotion estimation AI analysis unit. The AI analysis unit uses, for example, multimodal Transformers or ResNet+BERT ensemble models to output emotion labels (e.g., stress, fatigue, relaxation) and emotion intensity scores. Input examples include (1) voice saying “I'm tired”+expressionless image, (2) SNS post saying “I was able to relax today”+smiling image, etc. AI output examples include (1) emotion labels (stress, fatigue, relaxation), (2) emotion intensity score (0.0-1.0), etc. Based on these emotion estimation results, the acquisition unit applies health data priority determination logic (e.g., prioritize heart rate variability and skin conductance during stress, prioritize electromyography and sleep depth during fatigue, prioritize respiration rate and blood pressure during relaxation) and acquires high-priority data from health devices via Bluetooth or Wi-Fi communication. Subsequent processing includes real-time recording of preferentially acquired data and passing data to the health indicator fluctuation analysis or abnormality detection AI analysis unit by emotional state. This configuration prevents omission of important health data and improves analysis accuracy and reduces user burden by combining high-precision emotion estimation by AI and dynamic priority control, unlike conventional uniform data acquisition or subjective human judgment. Application fields include stress management apps, mental health support, and remote medical monitoring.

The acquisition unit can preferentially acquire relevant data by considering the user's geographic location information when acquiring health data. For example, if the user is in a high-altitude area, the acquisition unit prioritizes acquisition of data related to altitude. If the user is in an urban area, the acquisition unit prioritizes acquisition of data related to air quality. If the user is by the sea, the acquisition unit prioritizes acquisition of data related to humidity. By considering the user's geographic location information, relevant data can be acquired preferentially. Specifically, the acquisition unit acquires the user's current location (e.g., latitude, longitude, altitude) in real time using GPS modules or Wi-Fi location estimation functions. The acquisition unit matches this location information with a geographic information database and adds environmental attributes (e.g., elevation, urban/suburban/coastal labels, weather data collaboration). Input examples include (1) latitude 35.6, longitude 139.7, altitude 50 m (urban area), (2) latitude 36.2, longitude 137.8, altitude 1500 m (high-altitude area), (3) latitude 34.9, longitude 139.8, altitude 5 m (coastal area), etc. Based on this information, the acquisition unit applies health data priority acquisition logic for each geographic attribute (e.g., prioritize oxygen saturation and heart rate in high-altitude areas, air quality and PM2.5 in urban areas, humidity and salt intake in coastal areas) and preferentially acquires relevant data from health devices and environmental sensors via Bluetooth or Wi-Fi communication. Subsequent processing includes recording acquired data with geographic attributes and passing data to the environmental factor and health condition correlation analysis or abnormality detection AI analysis unit. This configuration realizes health data collection optimized for environmental factors by combining AI-based geographic information collaboration and dynamic data priority control, unlike conventional uniform data acquisition or methods relying on human experience, thereby providing technical effects such as improved analysis accuracy and early detection of environmental risks. Application fields include health management for mountaineers, lifestyle disease prevention in urban areas, and health monitoring in coastal regions.

The acquisition unit can analyze the user's social media activity and acquire relevant data when acquiring health data. For example, if the user posts about feeling stressed on social media, the acquisition unit acquires data related to stress level. If the user posts about exercise on social media, the acquisition unit acquires data related to exercise. If the user posts about meals on social media, the acquisition unit acquires data related to meals. By analyzing the user's social media activity, relevant health data can be acquired. Specifically, the acquisition unit automatically acquires the latest post text (e.g., 100 tokens per post, up to 10 posts per day) using SNS APIs or web scraping modules with the user's permission. The acquisition unit performs normalization and tokenization in the preprocessing unit, extracts emotion and action words (e.g., stress, exercise, meal, fatigue, relaxation), and inputs the data to the text analysis AI analysis unit. The AI analysis unit uses, for example, large language models or BERT-based emotion and action classification models to output emotion labels (e.g., stress, exercise, meal, indifference) and action labels for each post. Input examples include (1) “I got stressed at work today,” (2) “Went for a morning run,” (3) “Made dinner with a new recipe,” etc. AI output examples include (1) emotion labels (stress, exercise, meal), (2) action labels (exercise, meal), (3) relevance score (0.0-1.0), etc. Based on these output results, the acquisition unit applies health data acquisition filtering logic (e.g., acquire heart rate and skin conductance during stress posts, acquire calorie consumption and electromyography during exercise posts, acquire blood glucose and calorie intake during meal posts) and acquires relevant data from health devices via Bluetooth or Wi-Fi communication. Subsequent processing includes time series correlation analysis between SNS activity and health data and passing data to the abnormality detection AI analysis unit. This configuration realizes health data collection tailored to the user's actual psychological and behavioral state by combining AI-based SNS text analysis and dynamic data acquisition control, unlike conventional user self-reporting or subjective human judgment, thereby providing technical effects such as improved analysis accuracy and faster abnormality detection. Application fields include mental health support, lifestyle disease prevention, and behavior management for athletes.

The generation unit can estimate the user's emotions and adjust the manner of expressing the meal plan based on the estimated emotions of the user. For example, if the user is feeling stressed, the generation unit suggests a meal plan that promotes relaxation. If the user is tired, the generation unit suggests a meal plan that replenishes energy. If the user is relaxed, the generation unit suggests a meal plan that can be enjoyed. By adjusting the manner of expressing the meal plan according to the user's emotions, more appropriate meal plans can be provided. Emotion estimation is realized using, for example, emotion engines or generative AI with emotion estimation functions. Generative AI may include text generation AI (e.g., LLM) or multimodal generative AI, but is not limited to these examples. Specifically, the generation unit collects multimodal information for emotion estimation, such as voice data (e.g., one minute of speech waveform, 16 kHz sampling, 960,000 samples), facial image data (e.g., 128×128 pixel RGB image), and text data (e.g., SNS post, about 100 tokens). The generation unit performs noise removal (e.g., spectral subtraction, face detection/cropping, text normalization) and feature extraction (e.g., MFCC acoustic features, facial expression vectors, BERT embedding vectors) in the preprocessing unit and inputs them to the emotion estimation AI analysis unit. The emotion estimation AI analysis unit uses multimodal Transformers that handle voice, image, and text integratively, ensemble models combining convolutional neural networks for voice, ResNet for facial images, and large language models for text. Examples of AI input include (1) speech waveform saying “I'm tired today”+facial image+text, (2) smiling facial image+SNS post saying “Fun!”, (3) expressionless facial image+post saying “I'm stressed”, etc. Examples of AI output include (1) emotion labels (e.g., stress, relaxation, fatigue, activity), (2) emotion intensity score (continuous value from 0.0 to 1.0), and (3) emotion change trend (e.g., stress increased over the past 24 hours). Based on these emotion estimation results, the generation unit applies meal plan expression optimization algorithms (e.g., emphasize “healing” and “warmth” during stress, emphasize “energy replenishment” and “recovery” during fatigue, emphasize “enjoyment” and “variety” during relaxation) and reflects them in the generation of meal plan descriptions and recipe suggestion texts. For example, during stress, “an evening meal with herbal tea to calm the mind”; during fatigue, “a breakfast rich in protein and vitamins”; during relaxation, “a creative menu using new ingredients” are automatically generated. These expressions are realized by inputting emotion labels, health condition, and meal plan requirements as prompts to text generation AI (large language models) and outputting natural language descriptions and suggestions. Output examples include (1) “Today's recommendation is a Japanese-style soup to heal the mind and body,” (2) “We suggest a chicken breast and broccoli salad ideal for recovery from fatigue,” (3) “For a relaxing weekend, how about homemade pizza for the family?” The generation unit passes these outputs to the user interface unit and executes subsequent processing to reflect them in meal plan display and recipe suggestion screens. This configuration realizes personalized meal suggestions that are attentive to the user's psychological state by combining high-precision emotion estimation by AI and dynamic expression optimization, unlike conventional uniform meal plan presentation or subjective human judgment, thereby providing technical effects such as improved acceptance of meal plans, increased continuation rate, and promotion of healthy behaviors. Application fields include personal health management apps, mental health support services, meal suggestions for nursing care facilities, and corporate welfare platforms.

The generation unit can adjust the level of detail of the plan based on the user's health goals when generating the meal plan. For example, if the user aims for dieting, the generation unit generates a detailed meal plan focusing on calorie restriction. If the user aims for muscle gain, the generation unit generates a detailed meal plan focusing on protein intake. If the user aims for health maintenance, the generation unit generates a detailed meal plan focusing on nutritional balance. By adjusting the level of detail of the plan based on the user's health goals, more effective meal plans can be provided. Specifically, the generation unit receives the user's health goals (e.g., dieting, muscle gain, health maintenance, lifestyle disease prevention) from the health data analysis unit and applies different meal plan detail control algorithms for each goal. The generation unit automatically adjusts the detail parameters of meal plan components (e.g., daily and per-meal calories, protein/fat/carbohydrate ratios, vitamin and mineral amounts, types of ingredients, cooking methods, intake timing) according to the health goal. For example, for dieting, the generation unit specifies calories, carbohydrate, and fat amounts per meal, restrictions on snacks and late-night meals, and recommendations for low-GI ingredients in detail. For muscle gain, the generation unit specifies protein intake (e.g., daily body weight×1.5 g), amino acid score, number of meals (e.g., five times a day), and intake timing before and after training in detail. For health maintenance, the generation unit specifies balanced nutrient ratios (e.g., PFC balance), diverse ingredient combinations, and recommendations for seasonal vegetables and fruits in detail. Examples of AI input include (1) user's health goal (e.g., dieting), (2) latest health status score (e.g., BMI 27.5, body fat percentage 25%), (3) past week of meal history (e.g., frequent skipping of breakfast, frequent snacking), etc. Examples of AI output include (1) detailed one-week meal plan (e.g., 1800 kcal per day, 400 kcal breakfast, 600 kcal lunch, 700 kcal dinner, 100 kcal snack), (2) nutritional breakdown for each meal (e.g., 25 g protein, 10 g fat, 50 g carbohydrate), (3) recommended ingredient list (e.g., chicken breast, broccoli, brown rice), (4) detailed instructions for cooking methods and intake timing, etc. The generation unit passes these outputs to the user interface unit and executes subsequent processing to automatically generate meal plan displays and recipe suggestion screens according to the level of detail. This configuration realizes improved goal achievement rate, nutritional management accuracy, and continuous health behavior support by combining AI-based health goal-linked detail control and personalized meal suggestions, unlike conventional uniform meal plan presentation or methods relying on human experience. Application fields include diet support apps, nutrition management for athletes, lifestyle disease prevention programs, and corporate health management services.

The generation unit can apply different generation algorithms according to the user's food allergies when generating the meal plan. For example, if the user has a nut allergy, the generation unit generates a meal plan excluding nuts. If the user has a dairy allergy, the generation unit generates a meal plan excluding dairy products. If the user has a gluten allergy, the generation unit generates a meal plan excluding gluten. By generating a meal plan according to the user's food allergies, safe and appropriate meals can be provided. Specifically, the generation unit acquires the user's food allergy information (e.g., nuts, dairy products, gluten, shellfish, eggs) from the health database or user profile and applies different meal plan generation algorithms for each allergen. The generation unit refers to allergen information for each ingredient and recipe in the ingredient and recipe databases (e.g., ingredient ID, allergen label, content) and executes filtering to automatically exclude allergenic ingredients. Examples of AI input include (1) user's allergy information (e.g., nut allergy), (2) health status score (e.g., BMI 22.0), (3) meal goal (e.g., 1800 kcal per day, 80 g protein), etc. Examples of AI output include (1) allergen-removed meal plan (e.g., one-week menu without nuts), (2) recommended substitute ingredient list (e.g., soy products instead of nuts), (3) allergy risk score (e.g., 0.0=no risk), etc. In addition to allergen removal filtering, the generation unit applies substitute ingredient suggestion algorithms (e.g., nutrient matching, taste/texture similarity scoring) to maintain nutritional balance and meal satisfaction even when allergenic ingredients are excluded. Output examples include (1) “Salad using roasted soybeans instead of nuts,” (2) “Soy milk cream pasta without dairy products,” (3) “Gluten-free rice flour bread,” etc. The generation unit passes these outputs to the user interface unit and executes subsequent processing to automatically generate meal plan displays and recipe suggestion screens with allergy information. This configuration realizes personalized meal plan generation that balances safety and nutrition and reduces allergy accident risk and improves user satisfaction by combining high-precision allergen filtering and substitute suggestions by AI, unlike conventional manual work or simple allergy exclusion. Application fields include allergy-friendly health management apps, meal management for school lunches and nursing care facilities, and nutrition management for athletes.

The generation unit can estimate the user's emotions and adjust the length of the meal plan based on the estimated emotions of the user. For example, if the user is feeling stressed, the generation unit suggests a short-term meal plan with quick effects. If the user is relaxed, the generation unit suggests a long-term meal plan. If the user is in a hurry, the generation unit suggests an immediate-effect meal plan. By adjusting the length of the meal plan according to the user's emotions, meal plans of more appropriate duration can be provided. Emotion estimation is realized using, for example, emotion engines or generative AI with emotion estimation functions. Generative AI may include text generation AI (e.g., LLM) or multimodal generative AI, but is not limited to these examples. Specifically, the generation unit collects multimodal information for emotion estimation, such as voice data, facial image data, and text data, performs noise removal and feature extraction in the preprocessing unit, and inputs them to the emotion estimation AI analysis unit. Examples of AI input include (1) voice saying “I'm tired today”+facial image+text, (2) SNS post saying “I was able to relax”+smiling image, (3) voice saying “I'm in a hurry”+anxious facial image, etc. Examples of AI output include (1) emotion labels (stress, relaxation, hurry), (2) emotion intensity score (0.0-1.0), etc. Based on these emotion estimation results, the generation unit applies meal plan duration optimization algorithms (e.g., three-day short-term intensive plan during stress, one-month long-term plan during relaxation, one-day immediate-effect plan during hurry) and automatically adjusts the duration, content, and goal setting of the meal plan. Output examples include (1) “Three-day detox menu for refreshment,” (2) “One-month balanced health plan,” (3) “One-day immediate energy replenishment menu,” etc. The generation unit passes these outputs to the user interface unit and executes subsequent processing to automatically generate meal plan displays and recipe suggestion screens by duration. This configuration realizes flexible meal plan provision tailored to the user's psychological state and living conditions and improves continuation rate and promotes healthy behaviors by combining AI-based emotion estimation and dynamic duration optimization, unlike conventional uniform duration setting or subjective human judgment. Application fields include short-term intensive diet programs, long-term health maintenance plans, and pre-event physical condition management support.

The generation unit can determine the priority of the plan based on the timing of submission of the user's health data when generating the meal plan. For example, if the user has recently submitted health data, the generation unit generates the latest meal plan based on that data. If the user has submitted health data in the past, the generation unit generates a meal plan in line with long-term health goals based on that data. If the user submits health data before a specific event, the generation unit generates a meal plan tailored to that event. By determining the priority of the plan based on the timing of submission of the user's health data, meal plans reflecting the latest health condition can be provided. Specifically, the generation unit manages the submission date and time information of the user's health data (e.g., heart rate, blood pressure, body weight, sleep patterns) as metadata and applies different plan priority determination algorithms for each submission timing. When the latest data is submitted, the generation unit prioritizes generation of a meal plan optimized for real-time health condition; when past data is submitted, the generation unit generates a plan based on long-term trends and goal achievement; when data is submitted before an event, the generation unit generates a special plan tailored to the event (e.g., marathon, health checkup, travel). Examples of AI input include (1) health data submission date and time (e.g., 2024/6/1 08:00), (2) health status score (e.g., BMI 24.0), (3) event information (e.g., marathon participation on 6/10), etc. Examples of AI output include (1) plan reflecting latest health condition (e.g., calorie adjustment menu for recent weight gain), (2) plan for long-term goals (e.g., one-month body fat reduction plan), (3) event-specific plan (e.g., carb-loading menu before competition), etc. The generation unit passes these outputs to the user interface unit and executes subsequent processing such as meal plan display and reminder notifications by submission timing. This configuration realizes flexible plan provision responsive to changes in health condition, improved goal achievement rate, and enhanced event responsiveness by combining AI-based submission timing-linked priority control and personalized meal suggestions, unlike conventional uniform plan presentation or methods relying on human experience. Application fields include meal management before and after health checkups, nutrition plans for sports event participants, and long-term health maintenance programs.

The generation unit can adjust the order of the plan based on relevance when generating the meal plan. For example, if the user prefers specific ingredients, the generation unit preferentially suggests meal plans including those ingredients. If the user prefers a specific meal style, the generation unit preferentially suggests meal plans in line with that style. If the user has specific health goals, the generation unit preferentially suggests meal plans in line with those goals. By adjusting the order of the plan based on relevance, more appropriate meal plans can be provided. Specifically, the generation unit acquires the user's preference information (e.g., preferred ingredients, disliked ingredients, preferred meal style, allergy information, health goals) from the user profile and applies relevance scoring algorithms (e.g., ingredient match score, style compatibility score, goal achievement score) to rank multiple meal plan candidates. The generation unit collaborates with ingredient and recipe databases and places plans matching the user's preferences at the top and plans with lower relevance at the bottom. Examples of AI input include (1) user's preferences (e.g., likes Japanese cuisine, likes chicken), (2) health goals (e.g., weight loss), (3) allergy information (e.g., no dairy products), etc. Examples of AI output include (1) meal plan list with relevance scores (e.g., Japanese cuisine-focused plan 90 points, Western cuisine-focused plan 60 points), (2) reason for recommendation (e.g., contains many preferred ingredients), (3) plan order (e.g., 1st Japanese, 2nd Chinese, 3rd Western), etc. The generation unit passes these outputs to the user interface unit and executes subsequent processing to automatically generate meal plan displays and user selection screens in order of relevance. This configuration realizes improved user satisfaction, reduced selection stress, and increased continuation rate of healthy behaviors by combining AI-based relevance scoring and dynamic order optimization, unlike conventional uniform plan presentation or subjective human judgment. Application fields include personalized health management apps, meal suggestions for nursing care facilities, and nutrition management for athletes.

The list generation unit can estimate the user's emotions and adjust the manner of expressing the shopping list based on the estimated emotions of the user. For example, when the user is feeling stressed, the list generation unit generates a simple and highly visible shopping list. When the user is relaxed, the list generation unit generates a shopping list containing detailed information. When the user is in a hurry, the list generation unit generates a shopping list that focuses on key points. By adjusting the manner of expressing the shopping list according to the user's emotions, a more appropriate shopping list can be provided. Emotion estimation is realized, for example, by using an emotion engine or a generative AI with emotion estimation functionality. The generative AI may be a text generation AI (e.g., LLM) or a multimodal generative AI, but is not limited to these examples. Specifically, the list generation unit collects multimodal information for emotion estimation, such as voice data (e.g., 1-minute speech waveform, 16 kHz sampling, 960,000 samples), facial image data (e.g., 128×128 pixel RGB image), and text data (e.g., SNS post, about 100 tokens). The list generation unit performs noise removal (e.g., spectral subtraction, face detection and cropping, text normalization) and feature extraction (e.g., MFCC acoustic features, facial expression vectors, BERT embedding vectors) in a preprocessing unit, and inputs the processed data to an emotion estimation AI analysis unit. The emotion estimation AI analysis unit uses an ensemble model combining a multimodal Transformer that integrally handles voice, image, and text, a convolutional neural network for voice, ResNet for facial images, and a large language model for text. Examples of AI input include (1) speech waveform of “I'm tired today”+facial image+text, (2) smiling facial image+SNS post “I'm happy!”, (3) expressionless facial image+post “I'm stressed out”. Examples of AI output include (1) emotion labels (e.g., stress, relaxation, fatigue, active), (2) emotion intensity scores (continuous values from 0.0 to 1.0), and (3) emotion change trends (e.g., stress increase over the past 24 hours). Based on these emotion estimation results, the list generation unit applies a shopping list expression optimization algorithm (e.g., during stress, limit the number of items, increase font size, clarify color coding; during relaxation, add detailed ingredient information, storage methods, recipe links; during hurry, display only essential ingredient names and quantities) to automatically adjust the content, layout, and amount of information in the shopping list. Examples of output include (1) during stress: display only “2 packs of eggs, 500 g chicken, 1 bunch of spinach” in large letters; (2) during relaxation: display detailed information such as “2 packs of eggs (expiration 6/15, refrigerated), 500 g chicken (domestic, 3 days storage), 1 bunch of spinach (recipe: ohitashi)”; (3) during hurry: list only “eggs, chicken, spinach”. The list generation unit passes these outputs to the user interface unit and executes subsequent processing to display them in the optimal format on smartphone apps or wearable device screens. This configuration, unlike conventional uniform shopping list presentation or subjective human judgment, combines high-precision emotion estimation by AI and dynamic list expression optimization to provide personalized shopping lists tailored to the user's psychological state and situation, improve shopping efficiency, reduce stress, and prevent information overload or deficiency. Application fields include personal shopping support apps, ingredient ordering for care facilities, corporate welfare services, and family-shared shopping list systems.

The list generation unit can adjust the level of detail of the shopping list based on the importance of the ingredients when generating the shopping list. For example, the list generation unit provides detailed information for major ingredients and concise information for supplementary ingredients. The list generation unit may also provide detailed information for ingredients that are frequently used, based on their usage frequency. Furthermore, the list generation unit may provide detailed information for ingredients that are difficult to store, based on their storage period. By adjusting the level of detail of the list according to the importance of the ingredients, more efficient shopping becomes possible. Specifically, the list generation unit is equipped with an algorithm that automatically calculates an importance score for each ingredient (e.g., major ingredient=100 points, supplementary ingredient=50 points, seasoning=20 points) in cooperation with an ingredient database (e.g., ingredient ID, category, storage period, usage frequency, nutritional value, inventory status). The list generation unit receives the required ingredient list from the meal plan generation unit as input data and refers to parameters for each ingredient such as (1) usage frequency (e.g., used 5 times a week), (2) storage period (e.g., eggs 14 days, spinach 2 days), (3) nutritional value (e.g., protein content), and (4) inventory status (e.g., 2 left in the refrigerator). Examples of input include (1) major ingredient: chicken (used 5 times a week, 3 days storage), (2) supplementary ingredient: parsley (used once a week, 7 days storage), (3) seasoning: salt (used 7 times a week, 1 year storage). Based on this information, the list generation unit applies a list detail optimization algorithm that adds detailed information such as origin, storage method, expiration date, and recommended brand for major ingredients, and records only concise names and required quantities for supplementary ingredients and seasonings. Examples of output include (1) major ingredient: “500 g chicken (domestic, 3 days storage, expiration 6/10)”, (2) supplementary ingredient: “1 bunch parsley”, (3) seasoning: “salt as needed”. The list generation unit passes these outputs to the user interface unit and executes subsequent processing such as displaying the shopping list or generating a printable list according to the level of detail. This configuration, unlike conventional uniform list descriptions or methods relying on human experience, combines AI-based ingredient importance scoring and dynamic detail control to improve shopping efficiency, reduce food waste, prevent information overload or deficiency, and enhance user satisfaction. Application fields include household shopping support apps, ingredient ordering for care facilities, inventory management for corporate cafeterias, and nutrition management for athletes.

The list generation unit can apply different generation algorithms according to the category of ingredients when generating the shopping list. For example, for fresh foods, the list generation unit generates a shopping list that emphasizes freshness. For processed foods, the list generation unit generates a shopping list that emphasizes storage period. For seasonings, the list generation unit generates a shopping list that emphasizes usage frequency. By applying generation algorithms according to the category of ingredients, a more appropriate shopping list can be provided. Specifically, the list generation unit refers to category information stored in the ingredient database (e.g., fresh foods, processed foods, seasonings, frozen foods, beverages, etc.) and automatically applies different list generation algorithms for each category (e.g., for fresh foods, emphasize purchase date, consumption deadline, and storage method; for processed foods, emphasize expiration date and inventory status; for seasonings, emphasize usage frequency and remaining quantity). The list generation unit receives the required ingredient list from the meal plan generation unit as input data and determines the category of each ingredient (e.g., eggs=fresh food, ham=processed food, salt=seasoning). Examples of input include (1) fresh food: spinach (consumption deadline 2 days), (2) processed food: bacon (expiration date 14 days), (3) seasoning: soy sauce (remaining 50 ml, used 3 times a week). Based on this information, the list generation unit determines the optimal information structure, order, and emphasis items for each category and automatically generates the shopping list. Examples of output include (1) fresh food: “1 bunch spinach (consumption deadline 6/8, refrigerated)”, (2) processed food: “100 g bacon (expiration 6/20)”, (3) seasoning: “soy sauce (remaining 50 ml, used 3 times a week)”. The list generation unit passes these outputs to the user interface unit and executes subsequent processing such as category-based display, color-coded display, and category-based printable list generation. This configuration, unlike conventional uniform list descriptions or subjective human judgment, combines AI-based category determination and dynamic algorithm application to improve shopping efficiency, reduce food waste, enhance inventory management accuracy, and improve user satisfaction. Application fields include household shopping support apps, ingredient ordering for care facilities, inventory management for corporate cafeterias, and nutrition management for athletes.

The list generation unit can estimate the user's emotions and adjust the length of the shopping list based on the estimated emotions of the user. For example, when the user is feeling stressed, the list generation unit generates a short shopping list that focuses on key points. When the user is relaxed, the list generation unit generates a longer shopping list containing detailed information. When the user is in a hurry, the list generation unit generates a short shopping list to enable quick shopping. By adjusting the length of the shopping list according to the user's emotions, a more appropriate shopping list can be provided. Emotion estimation is realized, for example, by using an emotion engine or a generative AI with emotion estimation functionality. The generative AI may be a text generation AI (e.g., LLM) or a multimodal generative AI, but is not limited to these examples. Specifically, the list generation unit collects multimodal information for emotion estimation, such as voice data (e.g., 1-minute speech waveform, 16 kHz sampling, 960,000 samples), facial image data (e.g., 128×128 pixel RGB image), and text data (e.g., SNS post, about 100 tokens). The list generation unit performs noise removal (e.g., spectral subtraction, face detection and cropping, text normalization) and feature extraction (e.g., MFCC acoustic features, facial expression vectors, BERT embedding vectors) in a preprocessing unit, and inputs the processed data to an emotion estimation AI analysis unit. The emotion estimation AI analysis unit uses an ensemble model combining a multimodal Transformer that integrally handles voice, image, and text, a convolutional neural network for voice, ResNet for facial images, and a large language model for text. Examples of AI input include (1) speech waveform of “I'm tired today”+facial image+text, (2) smiling facial image+SNS post “I'm happy!”, (3) expressionless facial image+post “I'm stressed out”. Examples of AI output include (1) emotion labels (e.g., stress, relaxation, hurry), (2) emotion intensity scores (continuous values from 0.0 to 1.0). Based on these emotion estimation results, the list generation unit applies a shopping list length optimization algorithm (e.g., during stress, extract only major ingredients and limit to 3-5 items; during relaxation, include supplementary ingredients, seasonings, and storage methods for 10 or more items; during hurry, display only the most important 2-3 ingredients) to automatically adjust the number of items, amount of information, and display order in the shopping list. Examples of output include (1) during stress: display only “2 packs of eggs, 500 g chicken, 1 bunch of spinach”; (2) during relaxation: display a detailed and longer list such as “2 packs of eggs (expiration 6/15), 500 g chicken (3 days storage), 1 bunch of spinach, 1 bunch parsley, 1 L milk, 100 g butter”; (3) during hurry: list only “eggs, chicken”. The list generation unit passes these outputs to the user interface unit and executes subsequent processing such as display, printing, or voice reading according to the list length. This configuration, unlike conventional uniform list presentation or subjective human judgment, combines high-precision emotion estimation by AI and dynamic list length optimization to provide personalized shopping lists tailored to the user's psychological state and situation, improve shopping efficiency, reduce stress, and prevent information overload or deficiency. Application fields include personal shopping support apps, ingredient ordering for care facilities, corporate welfare services, and family-shared shopping list systems.

The list generation unit can determine the priority of the shopping list based on the submission timing of the ingredients when generating the shopping list. For example, the list generation unit places ingredients that need to be purchased urgently at the top of the list. The list generation unit places ingredients that can be stored for a long time at the bottom of the list. The list generation unit places frequently used ingredients at the top of the list. By determining the priority of the list based on the submission timing of the ingredients, more efficient shopping becomes possible. Specifically, the list generation unit cooperates with an ingredient database and inventory management system to automatically acquire metadata such as the submission timing of each ingredient (e.g., meal plan generation date, out-of-stock prediction date, consumption deadline), scheduled usage date, possible storage period, and usage frequency. Based on this information, the list generation unit applies a list priority determination algorithm (e.g., order by nearest consumption deadline, earliest scheduled usage date, highest usage frequency, longest storage period) to automatically adjust the order of the shopping list. Examples of input include (1) eggs (consumption deadline 6/8, used 5 times a week), (2) chicken (consumption deadline 6/7, used 3 times a week), (3) salt (expiration 1 year, used 7 times a week). Based on this information, the list generation unit applies a priority optimization logic that places ingredients that need to be purchased urgently (e.g., near consumption deadline, almost out of stock) at the top of the list, ingredients that can be stored for a long time (e.g., salt, sugar, dried foods) at the bottom, and frequently used ingredients (e.g., eggs, milk) at the top. Examples of output include (1) 1st: 500 g chicken (consumption deadline 6/7), (2) 2nd: 2 packs of eggs (consumption deadline 6/8), (3) 3rd: salt (expiration 1 year). The list generation unit passes these outputs to the user interface unit and executes subsequent processing such as priority shopping list display, reminder notifications, and printable list generation. This configuration, unlike conventional uniform list descriptions or methods relying on human experience, combines AI-based submission timing, usage frequency, and storage period-linked priority control to improve shopping efficiency, reduce food waste, prevent out-of-stock situations, and enhance user satisfaction. Application fields include household shopping support apps, ingredient ordering for care facilities, inventory management for corporate cafeterias, and nutrition management for athletes.

The list generation unit can adjust the order of the shopping list based on the relevance of the ingredients when generating the shopping list. For example, the list generation unit groups ingredients of the same category together in the list. The list generation unit arranges ingredients in the list based on the order of use. The list generation unit arranges ingredients in the list based on storage method. By adjusting the order of the list according to the relevance of the ingredients, more efficient shopping becomes possible. Specifically, the list generation unit cooperates with an ingredient database and recipe database to automatically acquire attribute information for each ingredient, such as category (e.g., vegetables, meats, dairy products, seasonings), order of use (e.g., for breakfast, lunch, dinner), and storage method (e.g., refrigerated, frozen, room temperature). Based on this information, the list generation unit applies a list order optimization algorithm (e.g., category grouping, sorting by order of use, sorting by storage method) to automatically adjust the order of the shopping list. Examples of input include (1) vegetables: spinach, cabbage; (2) meats: chicken, pork; (3) dairy products: milk, yogurt; (4) seasonings: salt, soy sauce; (5) storage method: refrigerated=spinach, milk; frozen=pork; room temperature=salt, etc. Based on this information, the list generation unit groups ingredients of the same category together in the list and optimizes the order according to order of use or storage method. Examples of output include (1) by category: “Vegetables: spinach, cabbage/Meats: chicken, pork/Dairy products: milk, yogurt/Seasonings: salt, soy sauce”; (2) by order of use: “For breakfast: eggs, milk/For lunch: chicken, cabbage/For dinner: pork, spinach”; (3) by storage method: “Refrigerated: spinach, milk/Frozen: pork/Room temperature: salt, soy sauce”. The list generation unit passes these outputs to the user interface unit and executes subsequent processing such as relevance-ordered shopping list display, printing, or voice reading. This configuration, unlike conventional uniform list descriptions or subjective human judgment, combines AI-based relevance scoring and dynamic order optimization to improve shopping efficiency, prevent forgetting to buy items, enhance inventory management accuracy, and improve user satisfaction. Application fields include household shopping support apps, ingredient ordering for care facilities, inventory management for corporate cafeterias, and nutrition management for athletes.

The suggestion unit can estimate the user's emotions and adjust the manner of expressing suggestions based on the estimated emotions of the user. For example, when the user is feeling stressed, the suggestion unit provides simple and highly visible suggestions. When the user is relaxed, the suggestion unit provides suggestions containing detailed information. When the user is in a hurry, the suggestion unit provides suggestions that focus on key points. By adjusting the manner of expressing suggestions according to the user's emotions, more appropriate suggestions can be provided. Emotion estimation is realized, for example, by using an emotion engine or a generative AI with emotion estimation functionality. The generative AI may be a text generation AI (e.g., LLM) or a multimodal generative AI, but is not limited to these examples. Specifically, the suggestion unit collects multimodal information for emotion estimation, such as voice data (e.g., 1-minute speech waveform, 16 kHz sampling, 960,000 samples), facial image data (e.g., 128×128 pixel RGB image), and text data (e.g., SNS post, about 100 tokens). The suggestion unit performs noise removal (e.g., spectral subtraction, face detection and cropping, text normalization) and feature extraction (e.g., MFCC acoustic features, facial expression vectors, BERT embedding vectors) in a preprocessing unit, and inputs the processed data to an emotion estimation AI analysis unit. The emotion estimation AI analysis unit uses an ensemble model combining a multimodal Transformer that integrally handles voice, image, and text, a convolutional neural network for voice, ResNet for facial images, and a large language model for text. Examples of AI input include (1) speech waveform of “I'm tired today”+facial image+text, (2) smiling facial image+SNS post “I'm happy!”, (3) expressionless facial image+post “I'm stressed out”. Examples of AI output include (1) emotion labels (e.g., stress, relaxation, hurry), (2) emotion intensity scores (continuous values from 0.0 to 1.0), and (3) emotion change trends (e.g., stress increase over the past 24 hours). Based on these emotion estimation results, the suggestion unit applies a suggestion expression optimization algorithm (e.g., during stress, limit the number of items, increase font size, clarify color coding; during relaxation, add detailed information, supplementary explanations, and options; during hurry, present only key points in bullet points) to automatically adjust the content, layout, and amount of information in the suggestions. Examples of output include (1) during stress: display only “Recommended supermarket: Store A (10 minutes on foot, all ingredients available)” in large letters; (2) during relaxation: display detailed information such as “Store A (10 minutes on foot, eggs 198 yen, chicken in stock, discount coupon valid), Store B (15 minutes on foot, eggs 210 yen, chicken out of stock)”; (3) during hurry: list only “Store A (all ingredients available)”. The suggestion unit passes these outputs to the user interface unit and executes subsequent processing to display them in the optimal format on smartphone apps or wearable device screens. This configuration, unlike conventional uniform suggestion presentation or subjective human judgment, combines high-precision emotion estimation by AI and dynamic suggestion expression optimization to provide personalized suggestions tailored to the user's psychological state and situation, improve decision-making efficiency, reduce stress, and prevent information overload or deficiency. Application fields include personal shopping support apps, ingredient procurement suggestions for care facilities, corporate welfare services, and family-shared shopping suggestion systems.

The suggestion unit can adjust the level of detail of suggestions based on the user's shopping status when making suggestions. For example, when the user is able to go shopping, the suggestion unit provides detailed information about the optimal supermarket. When the user does not have time to shop, the suggestion unit provides detailed suggestions on how to use an online supermarket. When the user does not have time to cook, the suggestion unit provides detailed menu suggestions for food delivery. By adjusting the level of detail of suggestions according to the user's shopping status, more appropriate suggestions can be provided. Specifically, the suggestion unit acquires input data such as schedule information (calendar API integration), current location information (GPS), past shopping history, and health data (e.g., fatigue score) to determine the user's shopping status (e.g., able to shop, no time, unable to cook). Based on this information, the suggestion unit uses a shopping status determination AI analysis unit (e.g., decision tree, random forest, rule-based classifier) to classify the user's status as “able to shop,” “online supermarket recommended,” or “food delivery recommended.” Examples of AI input include (1) schedule “18:00-19:00 meeting,” current location “home,” past shopping history “once this week,” (2) health data “fatigue score 0.8,” (3) cooking time “less than 30 minutes.” Examples of AI output include (1) shopping status label (able to shop, no time, unable to cook), (2) recommended suggestion type (supermarket details, online supermarket details, food delivery details). Based on these outputs, the suggestion unit provides detailed information such as store name, price, distance, discount information, and inventory status for supermarket suggestions; detailed guidance on order procedures, available delivery times, payment methods, and alternative inventory suggestions for online supermarket suggestions; and detailed display of menu name, nutritional content, allergen information, and estimated delivery time for food delivery suggestions. Subsequent processing includes automatically generating suggestion screens according to the level of detail in the user interface unit, and executing user selection, order confirmation, and reminder notifications. This configuration, unlike conventional uniform suggestion presentation or methods relying on human experience, combines AI-based shopping status determination and dynamic detail control to provide optimal information tailored to the user's situation, improve decision-making efficiency, and reduce the burden of shopping and cooking. Application fields include personal shopping support apps, ingredient procurement suggestions for care facilities, corporate welfare services, and family-shared shopping suggestion systems.

The suggestion unit can apply different suggestion algorithms according to the user's schedule when making suggestions. For example, when the user is busy, the suggestion unit proposes menus that can be cooked in a short time. When the user has spare time, the suggestion unit proposes elaborate dishes. When the user has no time before a specific event, the suggestion unit proposes quick meal plans. By applying suggestion algorithms according to the user's schedule, more appropriate suggestions can be provided. Specifically, the suggestion unit acquires input data such as schedule information (e.g., list of appointments via calendar API integration, free time blocks, event information), health data (e.g., fatigue score), and past cooking history. Based on this information, the suggestion unit uses a schedule analysis AI analysis unit (e.g., time-series LSTM, rule-based classifier, decision tree) to estimate the user's disposable time and the degree of leeway before and after events. Examples of AI input include (1) today's schedule: 18:00-21:00 meeting, 30 minutes free time; (2) event “Marathon on 6/10”; (3) fatigue score 0.7. Examples of AI output include (1) schedule label (busy, spare time, before event), (2) recommended cooking time (15 minutes, 60 minutes, quick). Based on these outputs, the suggestion unit automatically selects and proposes quick recipes or microwave cooking menus for busy times, elaborate dishes with multiple steps or recipes using new ingredients for spare time, and energy-supplementing or easily digestible quick menus for before events. Subsequent processing includes generating suggestion screens in the user interface unit that indicate cooking time and event suitability, displaying recipe details, cooking procedures, required ingredient lists, and order integration. This configuration, unlike conventional uniform suggestion presentation or methods relying on human experience, combines AI-based schedule analysis and dynamic algorithm application to provide optimal suggestions tailored to the user's lifestyle and events, improve cooking efficiency, and support continued healthy behavior. Application fields include personal health management apps, nutrition suggestions for event participants, meal arrangements for care facilities, and corporate welfare services.

The suggestion unit can estimate the user's emotions and adjust the length of suggestions based on the estimated emotions of the user. For example, when the user is feeling stressed, the suggestion unit provides short suggestions that focus on key points. When the user is relaxed, the suggestion unit provides longer suggestions containing detailed information. When the user is in a hurry, the suggestion unit provides quick and concise suggestions. By adjusting the length of suggestions according to the user's emotions, more appropriate suggestions can be provided. Emotion estimation is realized, for example, by using an emotion engine or a generative AI with emotion estimation functionality. The generative AI may be a text generation AI (e.g., LLM) or a multimodal generative AI, but is not limited to these examples. Specifically, the suggestion unit collects multimodal information for emotion estimation, such as voice data (e.g., 1-minute speech waveform, 16 kHz sampling, 960,000 samples), facial image data (e.g., 128×128 pixel RGB image), and text data (e.g., SNS post, about 100 tokens). The suggestion unit performs noise removal (e.g., spectral subtraction, face detection and cropping, text normalization) and feature extraction (e.g., MFCC acoustic features, facial expression vectors, BERT embedding vectors) in a preprocessing unit, and inputs the processed data to an emotion estimation AI analysis unit. The emotion estimation AI analysis unit uses an ensemble model combining a multimodal Transformer that integrally handles voice, image, and text, a convolutional neural network for voice, ResNet for facial images, and a large language model for text. Examples of AI input include (1) speech waveform of “I'm tired today”+facial image+text, (2) smiling facial image+SNS post “I'm happy!”, (3) expressionless facial image+post “I'm stressed out”. Examples of AI output include (1) emotion labels (e.g., stress, relaxation, hurry), (2) emotion intensity scores (continuous values from 0.0 to 1.0). Based on these emotion estimation results, the suggestion unit applies a suggestion length optimization algorithm (e.g., during stress, limit to key points within 3 lines; during relaxation, include detailed explanations, supplementary information, and options for 10 or more lines; during hurry, provide concise suggestions within 1-2 lines) to automatically adjust the length, amount of information, and display order of the suggestion text. Examples of output include (1) during stress: display only “Store A (all ingredients available)”; (2) during relaxation: display a detailed and longer suggestion such as “Store A (10 minutes on foot, eggs 198 yen, chicken in stock, discount coupon valid), Store B (15 minutes on foot, eggs 210 yen, chicken out of stock, double points)”; (3) during hurry: list only “Store A (shortest)”. The suggestion unit passes these outputs to the user interface unit and executes subsequent processing such as suggestion display, voice reading, or printing according to the length. This configuration, unlike conventional uniform suggestion presentation or subjective human judgment, combines high-precision emotion estimation by AI and dynamic suggestion length optimization to provide personalized suggestions tailored to the user's psychological state and situation, improve decision-making efficiency, reduce stress, and prevent information overload or deficiency. Application fields include personal shopping support apps, ingredient procurement suggestions for care facilities, corporate welfare services, and family-shared shopping suggestion systems.

The suggestion unit can determine the priority of suggestions based on the submission timing of the user's shopping status when making suggestions. For example, when the user has recently shopped, the suggestion unit provides the latest suggestions based on that data. When the user has past shopping data, the suggestion unit provides long-term suggestions based on that data. When the user has shopped before a specific event, the suggestion unit provides suggestions tailored to that event. By determining the priority of suggestions based on the submission timing of the user's shopping status, more appropriate suggestions can be provided. Specifically, the suggestion unit automatically acquires the user's shopping history data (e.g., purchase date and time, store, purchased items, event-linked information) from the database and applies different suggestion priority determination algorithms for each submission timing (e.g., latest history priority, long-term trend analysis, event-specific). The suggestion unit uses a shopping history analysis AI analysis unit (e.g., time-series LSTM, rule-based classifier) to estimate the user's shopping trends and event linkage. Examples of AI input include (1) latest purchase date “2024/6/1”, (2) event “Marathon on 6/10”, (3) list of purchased items in the past month. Examples of AI output include (1) suggestion priority label (latest, long-term, event), (2) recommended suggestion content (e.g., supermarket suggestion based on latest inventory, long-term ingredient rotation suggestion, event-specific menu suggestion). Based on these outputs, the suggestion unit automatically generates suggestions optimized for inventory status and recent purchases for latest history, ingredient rotation and preserved food suggestions for long-term history, and special menus or bulk purchase suggestions for events. Subsequent processing includes displaying suggestions with priority, reminder notifications, and order integration in the user interface unit. This configuration, unlike conventional uniform suggestion presentation or methods relying on human experience, combines AI-based submission timing-linked priority control and personalized suggestion generation to provide flexible suggestions that respond to the user's shopping status and events, improve shopping efficiency, prevent out-of-stock situations, and enhance event responsiveness. Application fields include household shopping support apps, ingredient suggestions for event participants, ingredient procurement for care facilities, and corporate welfare services.

The suggestion unit can adjust the order of suggestions based on relevance to the user when making suggestions. For example, when the user prefers certain ingredients, the suggestion unit prioritizes suggestions that include those ingredients. When the user prefers a particular dietary style, the suggestion unit prioritizes suggestions that match that style. When the user has specific health goals, the suggestion unit prioritizes suggestions that align with those goals. By adjusting the order of suggestions according to relevance to the user, more appropriate suggestions can be provided. Specifically, the suggestion unit acquires preference information (e.g., favorite ingredients, disliked ingredients, preferred dietary styles, allergy information, health goals) from the user profile and applies a relevance scoring algorithm (e.g., ingredient match score, style suitability score, goal achievement score) to rank multiple suggestion candidates. The suggestion unit cooperates with ingredient and recipe databases to place suggestions that match the user's preferences at the top and those with lower relevance at the bottom. Examples of AI input include (1) user preferences (e.g., likes Japanese food, likes chicken), (2) health goals (e.g., weight loss), (3) allergy information (e.g., no dairy products). Examples of AI output include (1) suggestion list with relevance scores (e.g., Japanese food-focused supermarket suggestion 90 points, Western food-focused online supermarket suggestion 60 points), (2) recommended reason (e.g., contains many favorite ingredients), (3) suggestion order (e.g., 1st Japanese food supermarket, 2nd Chinese food online supermarket, 3rd Western food delivery). The suggestion unit passes these outputs to the user interface unit and executes subsequent processing such as relevance-ordered suggestion display and user selection screen generation. This configuration, unlike conventional uniform suggestion presentation or subjective human judgment, combines AI-based relevance scoring and dynamic order optimization to improve user satisfaction, reduce selection stress, and increase the continuity of healthy behaviors. Application fields include personalized health management apps, ingredient procurement suggestions for care facilities, nutrition management for athletes, and corporate welfare services.

The system according to the embodiment is not limited to the examples described above and can be variously modified, for example, as follows. Specifically, the system may be configured to distribute each module, such as the health data acquisition unit, analysis unit, meal plan generation unit, shopping list generation unit, and suggestion unit, in a microservices architecture, and realize real-time processing by linking cloud-based GPU parallel computing clusters and edge devices. The system can flexibly change and expand the types of health data and acquisition devices (e.g., new vital sensors, environmental sensors, smart home appliance integration), AI model architectures (e.g., graph neural networks, self-supervised learning models, reinforcement learning models), meal plan generation algorithms (e.g., evolutionary algorithms, Bayesian optimization), and user interfaces for the suggestion unit (e.g., voice dialogue, AR display, chatbot integration). Furthermore, it is possible to implement variations such as integrated analysis of health data from multiple users, optimization of meal plans for families or groups, and optimization of ingredient ordering for entire facilities. These extensions provide technical effects such as improved system scalability, significant increase in processing speed, improved analysis accuracy, cooperative optimization among multiple users, and reduced operational costs. Application fields include deployment to various health management and meal management systems for households, care facilities, companies, schools, sports teams, and more.

The acquisition unit can analyze the user's past health data and select an optimal acquisition method when acquiring the user's health data. For example, the acquisition unit can select a method to acquire data during the most stable time period based on the user's past health data. The acquisition unit can also select a method to acquire data during time periods when specific health indicators are likely to fluctuate. Furthermore, the acquisition unit can select a method to optimize the frequency of data acquisition. By analyzing the user's past health data, the optimal acquisition method can be selected. Specifically, the acquisition unit automatically acquires time-series health data accumulated for each user (e.g., heart rate: 168-dimensional vector per minute, blood pressure: daily 2-dimensional array, body weight: daily 1-dimensional array, sleep patterns: 7 days×8 hours array) from the database. The acquisition unit applies time-series analysis algorithms (e.g., autoregressive models, moving averages, seasonal decomposition, coefficient of variation calculation) to these data to quantitatively evaluate the stability and variability of each health indicator. Examples of input include (1) time-series heart rate data for the past 30 days, (2) blood pressure trends for the past month, (3) sleep depth array for the past week. The AI analysis unit outputs (1) stability scores (e.g., standard deviation, coefficient of variation), (2) fluctuation peak times (e.g., heart rate increases at 22:00 every day), and (3) optimal acquisition frequency (e.g., 3 times a day, every 2 hours) from these input data. Based on these output results, the acquisition unit automatically generates a health data acquisition scheduler and optimizes the timing and frequency of data acquisition from health devices via Bluetooth or Wi-Fi communication. Subsequent processing includes collecting health data in real time according to the optimized acquisition schedule and passing the data to an abnormal value detection or health condition estimation AI analysis unit. This configuration, unlike conventional acquisition based on human experience or fixed schedules, combines AI-based time-series analysis and dynamic scheduling to realize data acquisition optimized for each user's lifestyle and health condition, thereby improving data utility, abnormality detection accuracy, and efficiency of communication and computational resources. Application fields include personal health management apps, remote monitoring for medical institutions, and health status monitoring for care facilities.

The generation unit can estimate the user's emotions when analyzing the user's health data and adjust the manner of expressing the meal plan based on the estimated emotions of the user. For example, when the user is feeling stressed, the generation unit can propose a meal plan that helps the user relax. When the user is tired, the generation unit can propose a meal plan that helps replenish energy. When the user is relaxed, the generation unit can propose a meal plan that the user can enjoy. By adjusting the manner of expressing the meal plan according to the user's emotions, a more appropriate meal plan can be provided. Specifically, the generation unit collects multimodal information for emotion estimation, such as voice data (e.g., 1-minute speech waveform, 16 kHz sampling, 960,000 samples), facial image data (e.g., 128×128 pixel RGB image), and text data (e.g., SNS post, about 100 tokens). The generation unit performs noise removal (e.g., spectral subtraction, face detection and cropping, text normalization) and feature extraction (e.g., MFCC acoustic features, facial expression vectors, BERT embedding vectors) in a preprocessing unit, and inputs the processed data to an emotion estimation AI analysis unit. The emotion estimation AI analysis unit uses an ensemble model combining a multimodal Transformer that integrally handles voice, image, and text, a convolutional neural network for voice, ResNet for facial images, and a large language model for text. Examples of AI input include (1) speech waveform of “I'm tired today”+facial image+text, (2) smiling facial image+SNS post “I'm happy!”, (3) expressionless facial image+post “I'm stressed out”. Examples of AI output include (1) emotion labels (e.g., stress, relaxation, fatigue, active), (2) emotion intensity scores (continuous values from 0.0 to 1.0), and (3) emotion change trends (e.g., stress increase over the past 24 hours). Based on these emotion estimation results, the generation unit applies a meal plan expression optimization algorithm (e.g., during stress, emphasize “healing” and “warmth”; during fatigue, emphasize “energy replenishment” and “recovery”; during relaxation, emphasize “enjoyment” and “variety”) and reflects these in the generation of meal plan descriptions and recipe proposals. For example, during stress, automatically generate expressions such as “Dinner with herbal tea to calm the mind”; during fatigue, “Propose a salad of chicken breast and broccoli optimal for recovery”; during relaxation, “How about homemade pizza for the family to enjoy on a relaxing weekend?” These expressions are realized by inputting emotion labels, health condition, and meal plan requirements as prompts to a text generation AI (large language model) and outputting natural language descriptions and proposals. Examples of output include (1) “Today's recommendation is a Japanese-style soup to heal body and mind”, (2) “We propose a chicken breast and broccoli salad optimal for fatigue recovery”, (3) “For a relaxing weekend, how about homemade pizza for the family?” The generation unit passes these outputs to the user interface unit and executes subsequent processing to reflect them in meal plan displays and recipe proposal screens. This configuration, unlike conventional uniform meal plan presentation or subjective human judgment, combines high-precision emotion estimation by AI and dynamic expression optimization to realize personalized meal proposals that are attentive to the user's psychological state, improve acceptance and continuity of meal plans, and promote healthy behaviors. Application fields include personal health management apps, mental health support services, meal proposals for care facilities, and corporate welfare platforms.

The suggestion unit can adjust the level of detail of suggestions based on the user's shopping status. For example, when the user is able to go shopping, the suggestion unit can provide detailed information about the optimal supermarket. When the user does not have time to shop, the suggestion unit can provide detailed suggestions on how to use an online supermarket. When the user does not have time to cook, the suggestion unit can provide detailed menu suggestions for food delivery. By adjusting the level of detail of suggestions according to the user's shopping status, more appropriate suggestions can be provided. Specifically, the suggestion unit automatically acquires various input data such as schedule information (e.g., list of appointments via calendar API integration, free time blocks), current location information (e.g., GPS coordinates), health data (e.g., fatigue score, activity level), and past shopping history (e.g., recent shopping date, store, purchased item list) to determine the user's shopping status. The suggestion unit performs normalization and feature extraction (e.g., generating free time vectors from schedule, calculating store distance from GPS, calculating activity score from health data) in a preprocessing unit and inputs the processed data to a shopping status determination AI analysis unit. The shopping status determination AI analysis unit uses, for example, decision trees, random forests, rule-based classifiers, or time-series LSTM models to classify the user's status into multiple labels such as “able to shop,” “online supermarket recommended,” or “food delivery recommended.” Examples of AI input include (1) schedule “18:00-19:00 meeting,” current location “home,” fatigue score 0.8; (2) past shopping history “once this week”; (3) activity level “5,000 steps today.” Examples of AI output include (1) shopping status label (able to shop, no time, unable to cook), (2) recommended suggestion type (supermarket details, online supermarket details, food delivery details). Based on these outputs, the suggestion unit provides detailed information such as store name, price, distance, discount information, and inventory status for supermarket suggestions; detailed guidance on order procedures, available delivery times, payment methods, and alternative inventory suggestions for online supermarket suggestions; and detailed display of menu name, nutritional content, allergen information, and estimated delivery time for food delivery suggestions. Subsequent processing includes automatically generating suggestion screens according to the level of detail in the user interface unit, and executing user selection, order confirmation, and reminder notifications. This configuration, unlike conventional uniform suggestion presentation or methods relying on human experience, combines AI-based shopping status determination and dynamic detail control to provide optimal information tailored to the user's situation, improve decision-making efficiency, and reduce the burden of shopping and cooking. Application fields include personal shopping support apps, ingredient procurement suggestions for care facilities, corporate welfare services, and family-shared shopping suggestion systems.

The list generation unit can estimate the user's emotions and adjust the manner of expressing the shopping list based on the estimated emotions of the user. For example, when the user is feeling stressed, the list generation unit can generate a simple and highly visible shopping list. When the user is relaxed, the list generation unit can generate a shopping list containing detailed information. When the user is in a hurry, the list generation unit can generate a shopping list that focuses on key points. By adjusting the manner of expressing the shopping list according to the user's emotions, a more appropriate shopping list can be provided. Specifically, the list generation unit collects multimodal information for emotion estimation, such as voice data (e.g., 1-minute speech waveform, 16 kHz sampling, 960,000 samples), facial image data (e.g., 128×128 pixel RGB image), and text data (e.g., SNS post, about 100 tokens). The list generation unit performs noise removal (e.g., spectral subtraction, face detection and cropping, text normalization) and feature extraction (e.g., MFCC acoustic features, facial expression vectors, BERT embedding vectors) in a preprocessing unit, and inputs the processed data to an emotion estimation AI analysis unit. The emotion estimation AI analysis unit uses an ensemble model combining a multimodal Transformer that integrally handles voice, image, and text, a convolutional neural network for voice, ResNet for facial images, and a large language model for text. Examples of AI input include (1) speech waveform of “I'm tired today”+facial image+text, (2) smiling facial image+SNS post “I'm happy!”, (3) expressionless facial image+post “I'm stressed out”. Examples of AI output include (1) emotion labels (e.g., stress, relaxation, fatigue, active), (2) emotion intensity scores (continuous values from 0.0 to 1.0), and (3) emotion change trends (e.g., stress increase over the past 24 hours). Based on these emotion estimation results, the list generation unit applies a shopping list expression optimization algorithm (e.g., during stress, limit the number of items, increase font size, clarify color coding; during relaxation, add detailed ingredient information, storage methods, recipe links; during hurry, display only essential ingredient names and quantities) to automatically adjust the content, layout, and amount of information in the shopping list. Examples of output include (1) during stress: display only “2 packs of eggs, 500 g chicken, 1 bunch of spinach” in large letters; (2) during relaxation: display detailed information such as “2 packs of eggs (expiration 6/15, refrigerated), 500 g chicken (domestic, 3 days storage), 1 bunch of spinach (recipe: ohitashi)”; (3) during hurry: list only “eggs, chicken, spinach”. The list generation unit passes these outputs to the user interface unit and executes subsequent processing to display them in the optimal format on smartphone apps or wearable device screens. This configuration, unlike conventional uniform shopping list presentation or subjective human judgment, combines high-precision emotion estimation by AI and dynamic list expression optimization to provide personalized shopping lists tailored to the user's psychological state and situation, improve shopping efficiency, reduce stress, and prevent information overload or deficiency. Application fields include personal shopping support apps, ingredient ordering for care facilities, corporate welfare services, and family-shared shopping list systems.

The acquisition unit can preferentially acquire relevant data by considering the user's geographic location information when acquiring health data. For example, when the user is in a high-altitude area, the acquisition unit can preferentially acquire data related to altitude. When the user is in an urban area, the acquisition unit can preferentially acquire data related to air quality. When the user is at the seaside, the acquisition unit can preferentially acquire data related to humidity. By considering the user's geographic location information, more relevant data can be preferentially acquired. Specifically, the acquisition unit acquires the user's current location (e.g., latitude, longitude, altitude) in real time using a GPS module or Wi-Fi location estimation function. The acquisition unit matches this location information with a geographic information database and adds environmental attributes (e.g., elevation, urban/suburban/seaside label, weather data integration). Examples of input include (1) latitude 35.6, longitude 139.7, altitude 50 m (urban area); (2) latitude 36.2, longitude 137.8, altitude 1500 m (high-altitude area); (3) latitude 34.9, longitude 139.8, altitude 5 m (seaside). Based on this information, the acquisition unit applies a health data priority acquisition logic for each geographic attribute (e.g., in high-altitude areas, prioritize oxygen saturation and heart rate; in urban areas, prioritize air quality and PM2.5; at the seaside, prioritize humidity and salt intake) and preferentially acquires relevant data from health devices or environmental sensors via Bluetooth or Wi-Fi communication. Subsequent processing includes recording the acquired data with geographic attributes and passing the data to correlation analysis of environmental factors and health status or abnormality detection AI analysis unit. This configuration, unlike conventional uniform data acquisition or methods relying on human experience, combines AI-based geographic information integration and dynamic data priority control to realize health data collection optimized for environmental factors, improve analysis accuracy, and enable early detection of environmental risks. Application fields include health management for mountaineers, prevention of lifestyle diseases in urban areas, and health monitoring in seaside regions.

The generation unit can adjust the level of detail of the meal plan based on the user's health goals when generating the meal plan. For example, when the user aims for weight loss, the generation unit can generate a detailed meal plan that emphasizes calorie restriction. When the user aims for muscle gain, the generation unit can generate a detailed meal plan that emphasizes protein intake. When the user aims for health maintenance, the generation unit can generate a detailed meal plan that emphasizes balance. By adjusting the level of detail of the plan according to the user's health goals, a more effective meal plan can be provided. Specifically, the generation unit receives the user's health goals (e.g., weight loss, muscle gain, health maintenance, prevention of lifestyle diseases) from the health data analysis unit and applies a different meal plan detail control algorithm for each goal. The generation unit automatically adjusts detail parameters for meal plan components (e.g., daily and per-meal calories, protein/fat/carbohydrate ratios, vitamin and mineral amounts, types of ingredients, cooking methods, timing of intake) according to the health goal. For example, for weight loss, the generation unit specifies calories, carbohydrate and fat amounts per meal, restrictions on snacks and late-night meals, and recommendations for low-GI ingredients in detail. For muscle gain, the generation unit specifies protein intake (e.g., body weight×1.5 g per day), amino acid score, number of meals (e.g., 5 times a day), and timing of intake before and after training in detail. For health maintenance, the generation unit specifies balanced nutrient ratios (e.g., PFC balance), combinations of diverse ingredients, and recommendations for seasonal vegetables and fruits in detail. Examples of AI input include (1) user's health goal (e.g., weight loss), (2) latest health status score (e.g., BMI 27.5, body fat percentage 25%), (3) past week's meal history (e.g., frequent skipping of breakfast, frequent snacking). Examples of AI output include (1) detailed meal plan for one week (e.g., 1800 kcal per day, 400 kcal breakfast, 600 kcal lunch, 700 kcal dinner, 100 kcal snack), (2) breakdown of nutrients for each meal (e.g., 25 g protein, 10 g fat, 50 g carbohydrates), (3) recommended ingredient list (e.g., chicken breast, broccoli, brown rice), (4) detailed instructions for cooking methods and timing of intake. The generation unit passes these outputs to the user interface unit and executes subsequent processing such as automatic generation of meal plan displays and recipe proposal screens according to the level of detail. This configuration, unlike conventional uniform meal plan presentation or methods relying on human experience, combines AI-based health goal-linked detail control and personalized meal proposals to improve goal achievement rate, nutrition management accuracy, and continuous support for healthy behaviors. Application fields include diet support apps, nutrition management for athletes, lifestyle disease prevention programs, and corporate health management services.

The suggestion unit can estimate the user's emotions and adjust the manner of expressing suggestions based on the estimated emotions of the user. For example, when the user is feeling stressed, the suggestion unit can provide simple and highly visible suggestions. When the user is relaxed, the suggestion unit can provide suggestions containing detailed information. When the user is in a hurry, the suggestion unit can provide suggestions that focus on key points. By adjusting the manner of expressing suggestions according to the user's emotions, a more appropriate suggestion can be provided. Specifically, the suggestion unit collects multimodal information for emotion estimation, such as voice data (e.g., 1-minute speech waveform, 16 kHz sampling, 960,000 samples), facial image data (e.g., 128×128 pixel RGB image), and text data (e.g., SNS post, about 100 tokens). The suggestion unit performs noise removal (e.g., spectral subtraction, face detection and cropping, text normalization) and feature extraction (e.g., MFCC acoustic features, facial expression vectors, BERT embedding vectors) in a preprocessing unit, and inputs the processed data to an emotion estimation AI analysis unit. The emotion estimation AI analysis unit uses an ensemble model combining a multimodal Transformer that integrally handles voice, image, and text, a convolutional neural network for voice, ResNet for facial images, and a large language model for text. Examples of AI input include (1) speech waveform of “I'm tired today”+facial image+text, (2) smiling facial image+SNS post “I'm happy!”, (3) expressionless facial image+post “I'm stressed out”. Examples of AI output include (1) emotion labels (e.g., stress, relaxation, hurry), (2) emotion intensity scores (continuous values from 0.0 to 1.0), and (3) emotion change trends (e.g., stress increase over the past 24 hours). Based on these emotion estimation results, the suggestion unit applies a suggestion expression optimization algorithm (e.g., during stress, limit the number of items, increase font size, clarify color coding; during relaxation, add detailed information, supplementary explanations, and options; during hurry, present only key points in bullet points) to automatically adjust the content, layout, and amount of information in the suggestions. Examples of output include (1) during stress: display only “Recommended supermarket: Store A (10 minutes on foot, all ingredients available)” in large letters; (2) during relaxation: display detailed information such as “Store A (10 minutes on foot, eggs 198 yen, chicken in stock, discount coupon valid), Store B (15 minutes on foot, eggs 210 yen, chicken out of stock)”; (3) during hurry: list only “Store A (all ingredients available)”. The suggestion unit passes these outputs to the user interface unit and executes subsequent processing to display them in the optimal format on smartphone apps or wearable device screens. This configuration, unlike conventional uniform suggestion presentation or subjective human judgment, combines high-precision emotion estimation by AI and dynamic suggestion expression optimization to provide personalized suggestions tailored to the user's psychological state and situation, improve decision-making efficiency, reduce stress, and prevent information overload or deficiency. Application fields include personal shopping support apps, ingredient procurement suggestions for care facilities, corporate welfare services, and family-shared shopping suggestion systems.

The acquisition unit can analyze the user's social media activity and acquire relevant data when acquiring health data. For example, if the user posts about feeling stressed on social media, data related to stress levels can be acquired. If the user posts about exercise, data related to exercise can also be acquired. Furthermore, if the user posts about meals, data related to meals can also be acquired. By analyzing the user's social media activity, relevant health data can be obtained. Specifically, the acquisition unit, with the user's permission, automatically acquires the latest post texts (e.g., 100 tokens per post, up to 10 posts per day) using SNS APIs or web scraping modules. The acquisition unit normalizes and tokenizes these text data in a preprocessing unit, extracts emotion and action words (e.g., stress, exercise, meal, fatigue, relaxation), and inputs them into a text analysis AI unit. The AI analysis unit, for example, uses large language models or BERT-based emotion and action classification models to output emotion labels (e.g., stress, exercise, meal, indifference) and action labels for each post. Examples of input include: (1) “I accumulated stress at work today,” (2) “I went running in the morning,” (3) “I cooked dinner with a new recipe.” Examples of AI output include: (1) emotion labels (stress, exercise, meal), (2) action labels (exercise, meal), (3) relevance scores (0.0-1.0), etc. Based on these output results, the acquisition unit applies health data acquisition filtering logic (e.g., for stress posts, acquires heart rate and skin conductance; for exercise posts, acquires calories burned and electromyography; for meal posts, acquires blood glucose and calorie intake) and acquires relevant data from health devices via Bluetooth or Wi-Fi communication. Subsequent processing includes time-series correlation analysis between SNS activity and health data, or passing data to an anomaly detection AI unit. This configuration, unlike conventional user self-reporting or subjective human judgment, combines AI-based SNS text analysis and dynamic data acquisition control to achieve health data collection tailored to the user's actual psychological and behavioral state, improved analysis accuracy, and faster anomaly detection. Application fields include mental health support, lifestyle disease prevention, and behavior management for athletes.

The generation unit can estimate the user's emotions and adjust the length of the meal plan based on the estimated emotions. For example, if the user is feeling stressed, a meal plan that produces results in a short period can be proposed. If the user is relaxed, a long-term meal plan can also be proposed. Furthermore, if the user is in a hurry, a meal plan with immediate effect can also be proposed. By adjusting the length of the meal plan according to the user's emotions, a more appropriate duration of the meal plan can be provided. Specifically, the generation unit collects multimodal information such as voice data, facial image data, and text data for emotion estimation, performs noise removal and feature extraction in a preprocessing unit, and inputs the data into an emotion estimation AI analysis unit. Examples of AI input include: (1) voice saying “I'm tired today”+facial image+text, (2) SNS post saying “I was able to relax”+smiling image, (3) voice saying “I'm in a hurry”+anxious facial image, etc. Examples of AI output include: (1) emotion labels (stress, relaxation, hurry), (2) emotion intensity scores (0.0-1.0), etc. Based on these emotion estimation results, the generation unit applies a meal plan duration optimization algorithm (e.g., short-term intensive plan for stress: 3 days; long-term plan for relaxation: 1 month; one-day plan for hurry) and automatically adjusts the duration, content, and goal setting of the meal plan. Examples of output include: (1) “Detox menu for refreshing in 3 days,” (2) “Balanced health plan for 1 month,” (3) “Quick menu for energy replenishment in 1 day,” etc. The generation unit passes these outputs to the user interface unit and executes subsequent processing such as automatic generation of screens for displaying meal plans by duration and recipe suggestions. This configuration, unlike conventional uniform duration settings or subjective human judgment, combines AI-based emotion estimation and dynamic duration optimization to provide flexible meal plans tailored to the user's psychological state and lifestyle, improve continuation rates, and promote healthy behavior. Application fields include short-term intensive diet programs, long-term health maintenance plans, and pre-event physical condition management support.

The list generation unit can determine the priority of the shopping list based on the timing of submission of ingredients when generating the shopping list. For example, ingredients that need to be purchased urgently can be placed at the top of the list. Ingredients that can be stored for a long time can be placed at the bottom of the list. Furthermore, ingredients with high usage frequency can also be placed at the top of the list. By determining the priority of the list based on the timing of submission of ingredients, more efficient shopping becomes possible. Specifically, the list generation unit cooperates with ingredient databases and inventory management systems to automatically acquire metadata such as submission timing for each ingredient (e.g., meal plan generation date, out-of-stock prediction date, expiration date), scheduled usage date, storage period, and usage frequency. Based on this information, the list generation unit applies a list priority determination algorithm (e.g., order by closest expiration date, earliest scheduled usage date, highest usage frequency, longest storage period) and automatically adjusts the order of the shopping list. Examples of input include: (1) eggs (expiration date 6/8, used 5 times a week), (2) chicken (expiration date 6/7, used 3 times a week), (3) salt (expiration date 1 year, used 7 times a week), etc. Based on this information, the list generation unit applies a priority optimization logic that places ingredients that need to be purchased urgently (e.g., close to expiration, nearly out of stock) at the top of the list, ingredients that can be stored for a long time (e.g., salt, sugar, dried foods) at the bottom, and ingredients with high usage frequency (e.g., eggs, milk) at the top. Examples of output include: (1) 1st: chicken 500 g (expiration date 6/7), (2) 2nd: 2 packs of eggs (expiration date 6/8), (3) 3rd: salt (expiration date 1 year), etc. The list generation unit passes these outputs to the user interface unit and executes subsequent processing such as displaying the shopping list with priorities, sending reminder notifications, and generating printable lists. This configuration, unlike conventional uniform list entries or methods relying on human experience, combines AI-based priority control linked to submission timing, usage frequency, and storage period to achieve improved shopping efficiency, reduced food waste, prevention of out-of-stock situations, and increased user satisfaction. Application fields include household shopping support apps, ingredient ordering for care facilities, inventory management for company cafeterias, and nutrition management for athletes.

The following is a brief explanation of the processing flow of Example of the Embodiment. Specifically, the system is composed of multiple modules such as a health data acquisition unit, data analysis unit, meal plan generation unit, shopping list generation unit, and suggestion unit, and each module may be distributed on a microservices architecture. The system automatically acquires various data in real time from the user's wearable devices, smartphones, environmental sensors, etc., including heart rate, blood pressure, body weight, sleep patterns, activity level, location information, multimodal data for emotion estimation (voice, image, text), and social media posts. The acquired data undergoes noise removal, normalization, and feature extraction (e.g., MFCC acoustic features, facial expression vectors, BERT embedding vectors, time-series features, geographic attribute assignment, action label assignment) in a preprocessing unit and is input to the analysis unit. The analysis unit links multiple AI models, such as time-series analysis AI (e.g., LSTM, autoregressive models), emotion estimation AI (e.g., multimodal Transformer, ResNet+BERT ensemble), behavior recognition AI (e.g., time-series convolutional neural networks), and text analysis AI (e.g., large language models), to generate diverse outputs such as health condition scores, emotion labels, activity labels, anomaly detection results, health goal estimation, and food allergy determination. These outputs are passed to the meal plan generation unit, which uses evolutionary algorithms, Bayesian optimization, and rule-based generation logic to generate personalized meal plans based on multidimensional parameters such as health condition, goals, preferences, allergies, emotions, activity status, geographic information, and SNS activity. The generated meal plan is automatically converted by the list generation unit into a shopping list that considers ingredient types, quantities, storage methods, categories, submission timing, importance, and relevance, and the detail level, order, length, and priority of the list are adjusted by AI-based dynamic optimization algorithms. Furthermore, the suggestion unit comprehensively analyzes the user's emotions, shopping status, schedule, health goals, preferences, event information, etc., and provides personalized suggestions for optimal supermarkets, online supermarkets, food delivery, cooking methods, purchase timing, suggestion expressions, information volume, and display order. The outputs of each module are passed to the user interface unit, where subsequent processing such as optimal format display, notifications, voice readout, and printing is executed on smartphone apps, wearable devices, and web dashboards. This system, unlike conventional human experience-based or simple automation methods, achieves high-dimensional data analysis and dynamic optimization through the collaboration of multiple AI models, thereby providing technical effects such as improved accuracy in health management, meal management, and shopping support, reduced user burden, faster anomaly detection, resource efficiency, and personalized behavior support. Application fields include personal health management apps, health, meal, and shopping support for care facilities, corporate welfare services, nutrition management for athletes, and family-shared health management systems.
Step 1: The acquisition unit acquires health data. Health data includes heart rate, blood pressure, body weight, and sleep patterns. The acquisition unit measures heart rate using wearable devices and collects the data. It can also measure blood pressure using a blood pressure monitor, measure body weight using a scale, and measure sleep patterns using a sleep tracker. Step 2: The generation unit analyzes the data acquired by the acquisition unit and generates a meal plan according to the user's health condition, goals, preferences, and food allergies. The generation unit analyzes the data using AI and evaluates the user's health condition. Based on the user's goals, for example, if the user aims to lose weight, a low-calorie and nutritionally balanced meal plan is generated. Based on the user's preferences, a meal plan including favorite ingredients can also be generated. Furthermore, based on the user's food allergies, for example, if the user has a nut allergy, a meal plan excluding nuts is generated. Step 3: The list generation unit automatically generates a shopping list based on the meal plan generated by the generation unit. The list generation unit lists the types and quantities of necessary ingredients. This enables the user to efficiently purchase the necessary ingredients when shopping. Step 4: The suggestion unit makes optimal suggestions based on the shopping list generated by the list generation unit. If the user can go shopping, the suggestion unit suggests the optimal supermarket based on online flyers. If the user does not have time to shop, the suggestion unit arranges for ingredients to be purchased from an online supermarket and delivered to arrive when the user is about to start cooking. If the user does not have time to cook, the suggestion unit suggests food delivery in accordance with the meal plan. Specifically, the system automatically acquires various data in real time from wearable devices and smartphones, including heart rate, blood pressure, body weight, sleep patterns, activity level, location information, multimodal data for emotion estimation (voice, image, text), and SNS posts, and performs noise removal, normalization, and feature extraction (e.g., MFCC acoustic features, facial expression vectors, BERT embedding vectors, time-series features, geographic attribute assignment, action label assignment) in a preprocessing unit. The data analysis unit links multiple AI models, such as time-series analysis AI (e.g., LSTM, autoregressive models), emotion estimation AI (e.g., multimodal Transformer, ResNet+BERT ensemble), behavior recognition AI (e.g., time-series convolutional neural networks), and text analysis AI (e.g., large language models), to generate diverse outputs such as health condition scores, emotion labels, activity labels, anomaly detection results, health goal estimation, and food allergy determination. These outputs are passed to the meal plan generation unit, which uses evolutionary algorithms, Bayesian optimization, and rule-based generation logic to generate personalized meal plans based on multidimensional parameters such as health condition, goals, preferences, allergies, emotions, activity status, geographic information, and SNS activity. The list generation unit automatically converts the meal plan into a shopping list that considers ingredient types, quantities, storage methods, categories, submission timing, importance, and relevance, and the detail level, order, length, and priority of the list are adjusted by AI-based dynamic optimization algorithms. The suggestion unit comprehensively analyzes the user's emotions, shopping status, schedule, health goals, preferences, event information, etc., and provides personalized suggestions for optimal supermarkets, online supermarkets, food delivery, cooking methods, purchase timing, suggestion expressions, information volume, and display order. The outputs of each module are passed to the user interface unit, where subsequent processing such as optimal format display, notifications, voice readout, and printing is executed on smartphone apps, wearable devices, and web dashboards. This system, unlike conventional human experience-based or simple automation methods, achieves high-dimensional data analysis and dynamic optimization through the collaboration of multiple AI models, thereby providing technical effects such as improved accuracy in health management, meal management, and shopping support, reduced user burden, faster anomaly detection, resource efficiency, and personalized behavior support. Application fields include personal health management apps, health, meal, and shopping support for care facilities, corporate welfare services, nutrition management for athletes, and family-shared health management systems.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned acquisition unit, generation unit, list generation unit, and suggestion unit is implemented, for example, in at least one of the smart device 14 and the data processing apparatus 12. For example, the acquisition unit acquires health data using the camera 42 or microphone 38B of the smart device 14, and collects data via the control unit 46A. The generation unit is implemented, for example, by a specific processing unit 290 of the data processing apparatus 12, analyzes health data using AI, and generates a meal plan. The list generation unit is implemented, for example, by the specific processing unit 290 of the data processing apparatus 12, and automatically generates a shopping list based on the generated meal plan. The suggestion unit is implemented, for example, by the control unit 46A of the smart device 14, and makes optimal suggestions based on the shopping list. The correspondence between each unit and the device or control unit is not limited to the examples described above and various modifications are possible.

### Second Embodiment

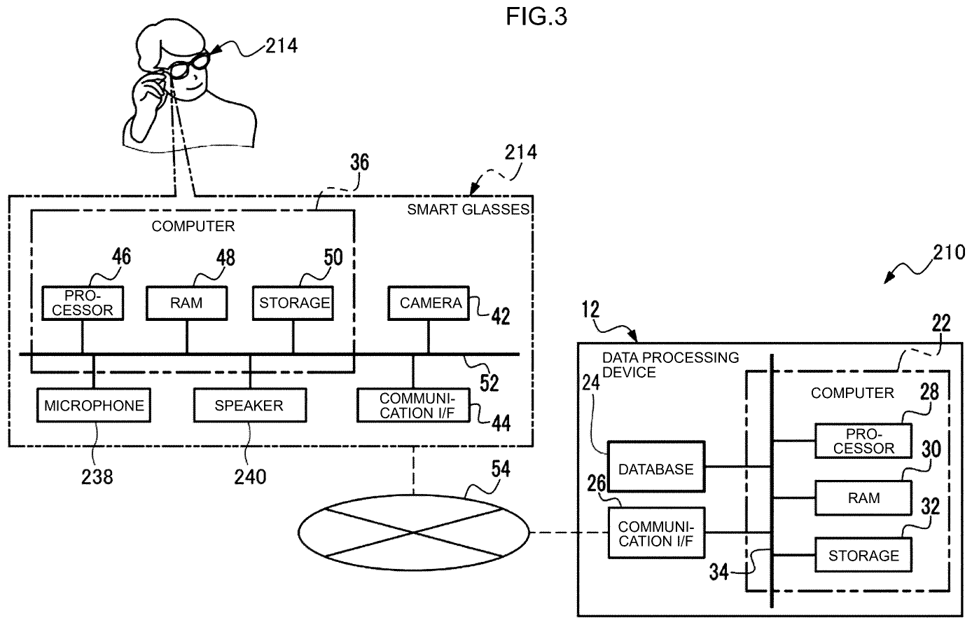

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

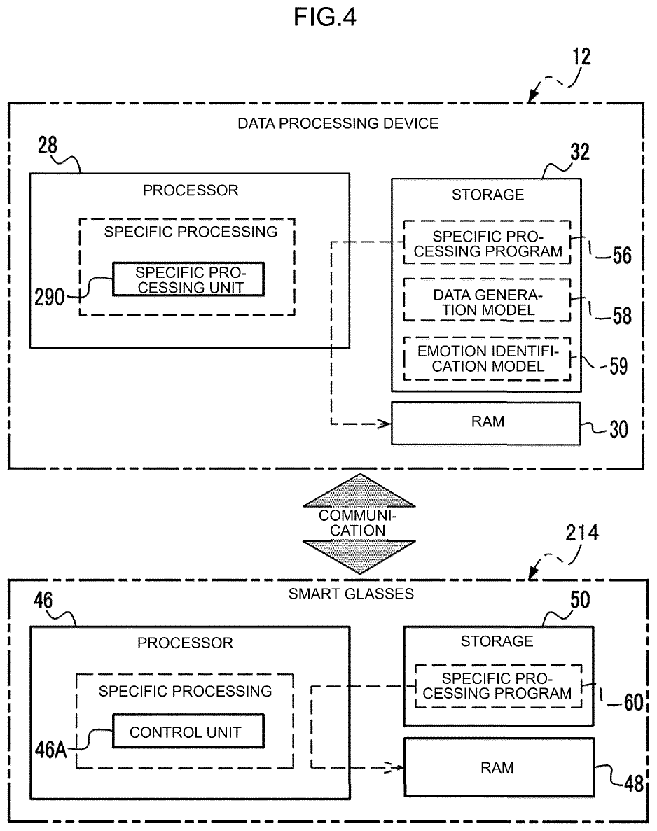

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned acquisition unit, generation unit, list generation unit, and suggestion unit is implemented, for example, in at least one of the smart glasses 214 and the data processing apparatus 12. For example, the acquisition unit acquires health data using the camera 42 or microphone 238 of the smart glasses 214, and collects data via the control unit 46A. The generation unit is implemented, for example, by a specific processing unit 290 of the data processing apparatus 12, analyzes health data using AI, and generates a meal plan. The list generation unit is implemented, for example, by the specific processing unit 290 of the data processing apparatus 12, and automatically generates a shopping list based on the generated meal plan. The suggestion unit is implemented, for example, by the control unit 46A of the smart glasses 214, and makes optimal suggestions based on the shopping list. The correspondence between each unit and the device or control unit is not limited to the examples described above and various modifications are possible.

### Third Embodiment

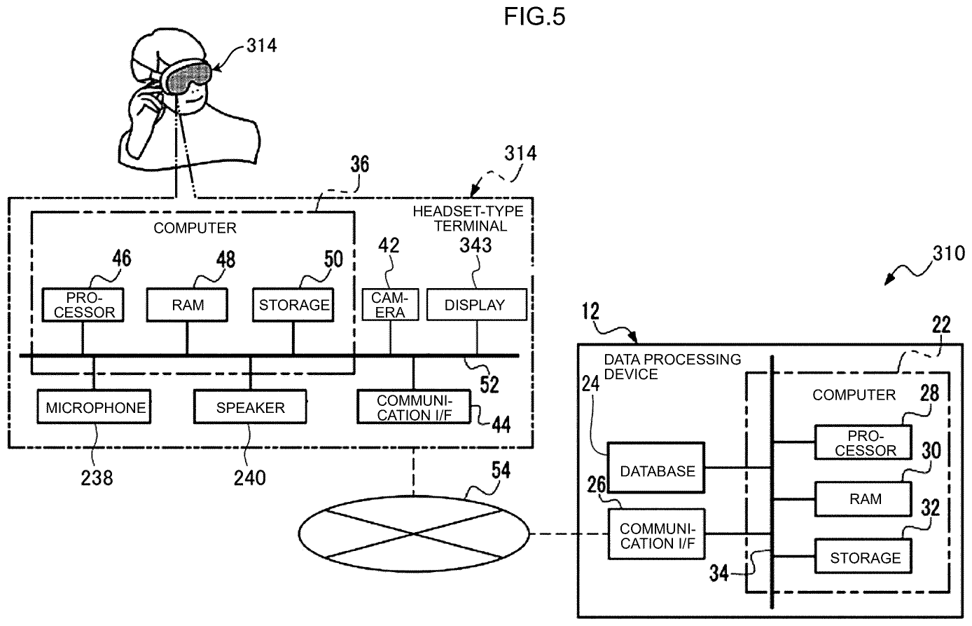

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

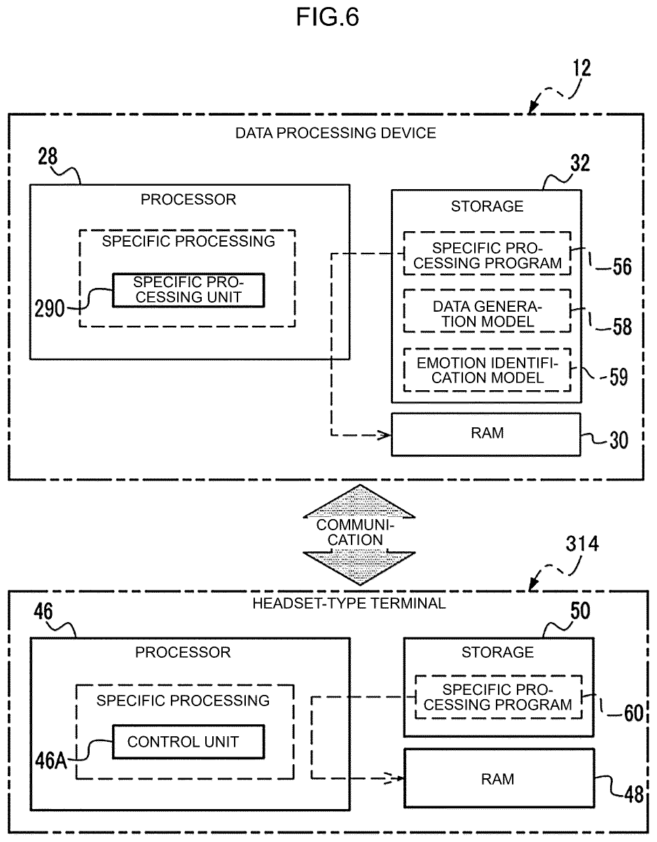

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned acquisition unit, generation unit, list generation unit, and suggestion unit is implemented, for example, in at least one of the headset-type terminal 314 and the data processing apparatus 12. For example, the acquisition unit acquires health data using the camera 42 or microphone 238 of the headset-type terminal 314, and collects data via the control unit 46A. The generation unit is implemented, for example, by a specific processing unit 290 of the data processing apparatus 12, analyzes health data using AI, and generates a meal plan. The list generation unit is implemented, for example, by the specific processing unit 290 of the data processing apparatus 12, and automatically generates a shopping list based on the generated meal plan. The suggestion unit is implemented, for example, by the control unit 46A of the headset-type terminal 314, and makes optimal suggestions based on the shopping list. The correspondence between each unit and the device or control unit is not limited to the examples described above and various modifications are possible.

### Fourth Embodiment

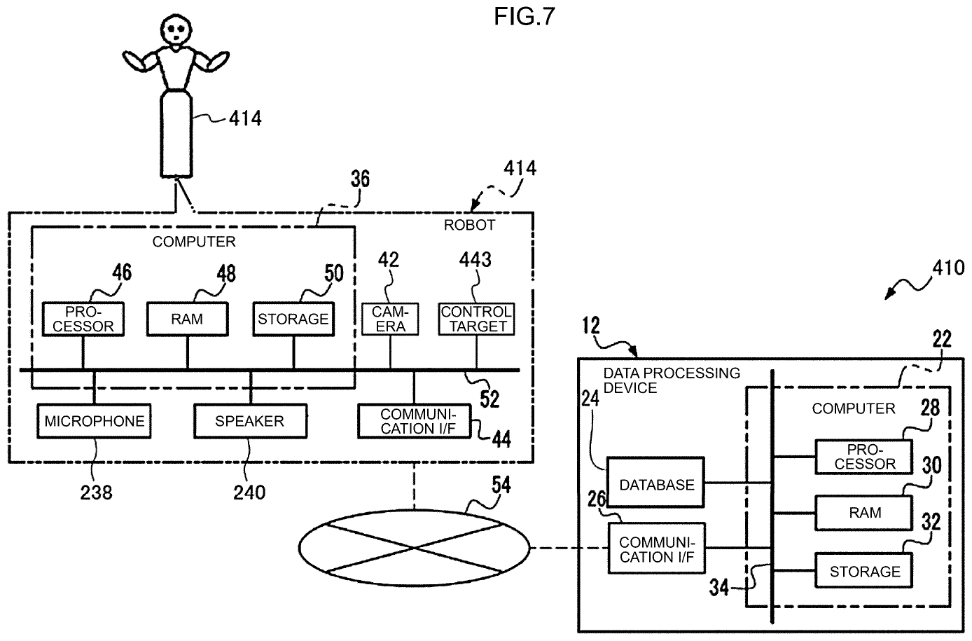

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

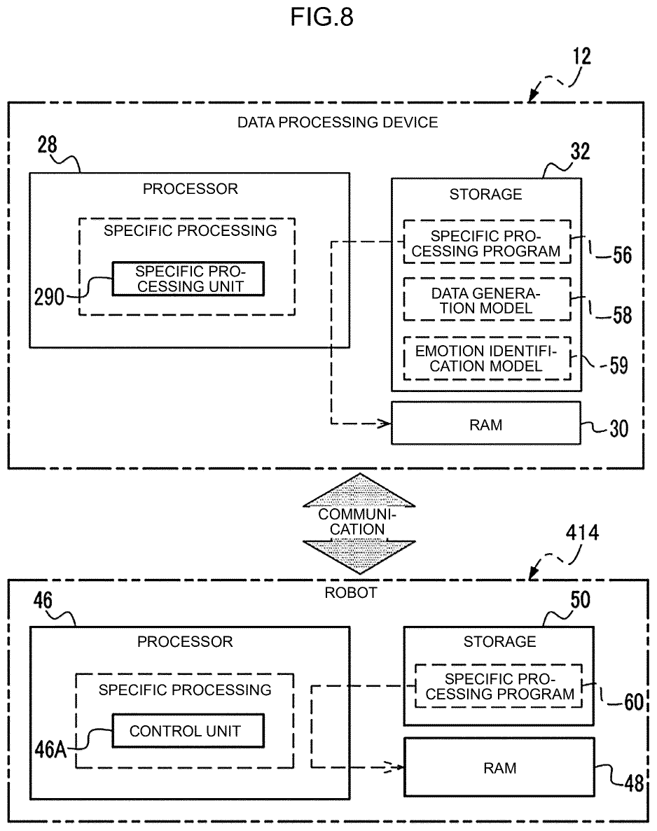

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned acquisition unit, generation unit, list generation unit, and suggestion unit is implemented, for example, in at least one of the robot 414 and the data processing apparatus 12. For example, the acquisition unit acquires health data using the camera 42 or microphone 238 of the robot 414, and collects data via the control unit 46A. The generation unit is implemented, for example, by a specific processing unit 290 of the data processing apparatus 12, analyzes health data using AI, and generates a meal plan. The list generation unit is implemented, for example, by the specific processing unit 290 of the data processing apparatus 12, and automatically generates a shopping list based on the generated meal plan. The suggestion unit is implemented, for example, by the control unit 46A of the robot 414, and makes optimal suggestions based on the shopping list. The correspondence between each unit and the device or control unit is not limited to the examples described above and various modifications are possible.

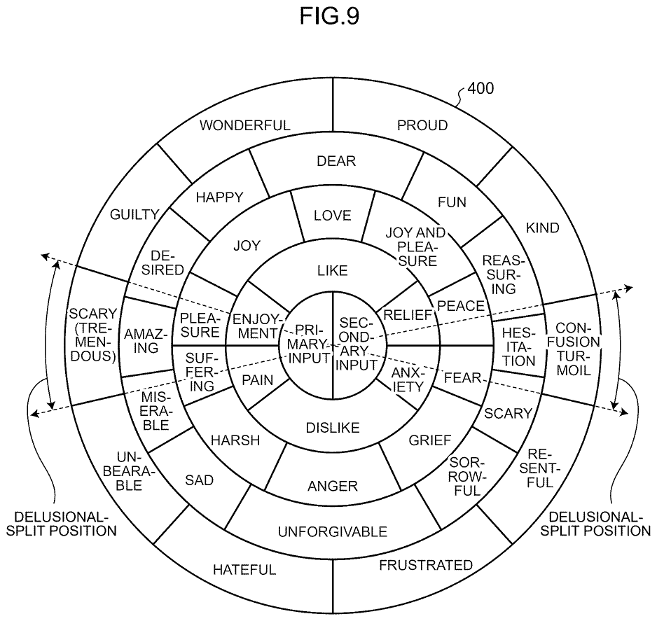

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

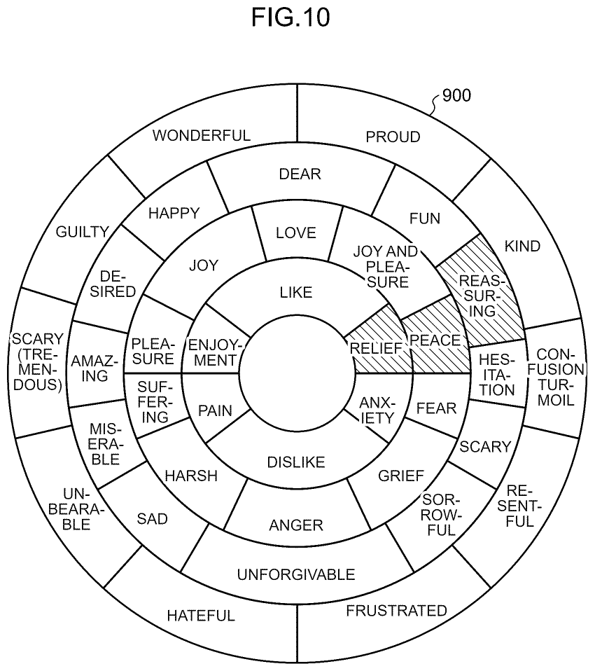

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: an acquisition unit configured to acquire health data; a generation unit configured to analyze the data acquired by the acquisition unit and generate a meal plan according to a user's health condition, goals, preferences, and food allergies; a list generation unit configured to automatically generate a shopping list based on the meal plan generated by the generation unit; and a suggestion unit configured to make suggestions based on the shopping list generated by the list generation unit.
(Supplementary Note 2) The system according to Supplementary Note 1, wherein the suggestion unit is configured to suggest a supermarket based on online flyers when the user is able to go shopping.
(Supplementary Note 3) The system according to Supplementary Note 1, wherein the suggestion unit is configured to arrange for ingredients to be purchased from an online supermarket and delivered to arrive when the user is about to start cooking, in cases where the user does not have time to go shopping.
(Supplementary Note 4) The system according to Supplementary Note 1, wherein the suggestion unit is configured to suggest food delivery in accordance with the meal plan when the user does not have time to cook.
(Supplementary Note 5) The system according to Supplementary Note 1, wherein the generation unit is configured to generate a menu that takes into account quantities that prevent food waste.
(Supplementary Note 6) The system according to Supplementary Note 1, wherein the acquisition unit is configured to acquire health data including heart rate, blood pressure, body weight, and sleep patterns.
(Supplementary Note 7) The system according to Supplementary Note 1, wherein the acquisition unit is configured to estimate the user's emotions and adjust the timing of health data acquisition based on the estimated emotions of the user.
(Supplementary Note 8) The system according to Supplementary Note 1, wherein the acquisition unit is configured to analyze the user's past health data and select an optimal acquisition method.
(Supplementary Note 9) The system according to Supplementary Note 1, wherein the acquisition unit is configured to perform filtering based on the user's current living conditions and activity level when acquiring health data.
(Supplementary Note 10) The system according to Supplementary Note 1, wherein the acquisition unit is configured to estimate the user's emotions and determine the priority of health data to be acquired based on the estimated emotions of the user.
(Supplementary Note 11) The system according to Supplementary Note 1, wherein the acquisition unit is configured to preferentially acquire relevant data by considering the user's geographic location information when acquiring health data.
(Supplementary Note 12) The system according to Supplementary Note 1, wherein the acquisition unit is configured to analyze the user's social media activity and acquire relevant data when acquiring health data.
(Supplementary Note 13) The system according to Supplementary Note 1, wherein the generation unit is configured to estimate the user's emotions and adjust the manner of expressing the meal plan based on the estimated emotions of the user.
(Supplementary Note 14) The system according to Supplementary Note 1, wherein the generation unit is configured to adjust the level of detail of the plan based on the user's health goals when generating the meal plan.
(Supplementary Note 15) The system according to Supplementary Note 1, wherein the generation unit is configured to apply different generation algorithms according to the user's food allergies when generating the meal plan.
(Supplementary Note 16) The system according to Supplementary Note 1, wherein the generation unit is configured to estimate the user's emotions and adjust the length of the meal plan based on the estimated emotions of the user.
(Supplementary Note 17) The system according to Supplementary Note 1, wherein the generation unit is configured to determine the priority of the plan based on the timing of submission of the user's health data when generating the meal plan.
(Supplementary Note 18) The system according to Supplementary Note 1, wherein the generation unit is configured to adjust the order of the plan based on relevance when generating the meal plan.
(Supplementary Note 19) The system according to Supplementary Note 1, wherein the list generation unit is configured to estimate the user's emotions and adjust the manner of expressing the shopping list based on the estimated emotions of the user.
(Supplementary Note 20) The system according to Supplementary Note 1, wherein the list generation unit is configured to adjust the level of detail of the list based on the importance of the ingredients when generating the shopping list.
(Supplementary Note 21) The system according to Supplementary Note 1, wherein the list generation unit is configured to apply different generation algorithms according to the category of ingredients when generating the shopping list.
(Supplementary Note 22) The system according to Supplementary Note 1, wherein the list generation unit is configured to estimate the user's emotions and adjust the length of the shopping list based on the estimated emotions of the user.
(Supplementary Note 23) The system according to Supplementary Note 1, wherein the list generation unit is configured to determine the priority of the list based on the timing of submission of the ingredients when generating the shopping list.
(Supplementary Note 24) The system according to Supplementary Note 1, wherein the list generation unit is configured to adjust the order of the list based on relevance when generating the shopping list.
(Supplementary Note 25) The system according to Supplementary Note 1, wherein the suggestion unit is configured to estimate the user's emotions and adjust the manner of expressing the suggestions based on the estimated emotions of the user.
(Supplementary Note 26) The system according to Supplementary Note 1, wherein the suggestion unit is configured to adjust the level of detail of the suggestions based on the user's shopping status when making suggestions.
(Supplementary Note 27) The system according to Supplementary Note 1, wherein the suggestion unit is configured to apply different suggestion algorithms according to the user's schedule when making suggestions.
(Supplementary Note 28) The system according to Supplementary Note 1, wherein the suggestion unit is configured to estimate the user's emotions and adjust the length of the suggestions based on the estimated emotions of the user.
(Supplementary Note 29) The system according to Supplementary Note 1, wherein the suggestion unit is configured to determine the priority of the suggestions based on the timing of submission of the user's shopping status when making suggestions.
(Supplementary Note 30) The system according to Supplementary Note 1, wherein the suggestion unit is configured to adjust the order of the suggestions based on relevance when making suggestions.

What is claimed is:

1. A system comprising:
circuitry configured to:
receive, via a communication interface coupled to a packet-switched network, sensor data transmitted from a client terminal, the sensor data representing a physiological state of a user captured by a sensor of the client terminal;
analyze the received sensor data by inputting the sensor data into a data generation model obtained by machine learning to generate plan data based on the physiological state, a goal, a preference, and a constraint of the user;
generate list data by extracting item identifiers and quantities from the plan data; and
generate recommendation data based on the list data and context data associated with the user, and transmit the recommendation data to the client terminal via the communication interface and the packet-switched network.

2. The system according to claim 1, wherein the sensor data comprises health data including at least one of heart rate data, blood pressure data, body weight data, or sleep pattern data captured by at least one of a wearable device, a blood pressure monitor, a scale, or a sleep tracker communicatively coupled to the client terminal.

3. The system according to claim 1, wherein the plan data comprises a meal plan generated according to the physiological state, the goal, the preference, and the constraint of the user, and wherein the constraint comprises a food allergy of the user.

4. The system according to claim 1, wherein the list data comprises a shopping list including ingredient names and required quantities extracted from the plan data, and wherein the circuitry is further configured to integrate duplicate items and adjust the list data based on an inventory status.

5. The system according to claim 1, wherein the circuitry is further configured to generate the recommendation data by applying a multivariate scoring algorithm to evaluate a plurality of candidate resources based on the context data, the context data comprising at least one of location information, schedule information, or pricing information associated with the user.

6. The system according to claim 1, wherein the circuitry is further configured to generate the recommendation data comprising an automated procurement arrangement by communicating with an external service via an application programming interface, the automated procurement arrangement including a delivery time optimized based on a schedule of the user.

7. The system according to claim 1, wherein the circuitry is further configured to generate the recommendation data by filtering and scoring a plurality of delivery options based on the plan data and the constraint of the user, and ranking the delivery options according to a compatibility score.

8. The system according to claim 1, wherein the circuitry is further configured to generate the plan data by applying an optimization algorithm to minimize surplus items across a multi-day period, the optimization algorithm comprising at least one of integer programming, graph search, or dynamic programming applied to item usage schedules and storage duration constraints.

9. The system according to claim 1, wherein the circuitry is further configured to estimate an emotion of the user based on multimodal input data received from the client terminal, and adjust a manner of expressing the plan data based on the estimated emotion, such that when the estimated emotion indicates stress, the circuitry generates the plan data in a first expression style, and when the estimated emotion indicates relaxation, the circuitry generates the plan data in a second expression style different from the first expression style.

10. The system according to claim 1, wherein the circuitry is further configured to estimate an emotion of the user by inputting multimodal input data into an emotion identification model obtained by machine learning, and adjust a timing of acquiring the sensor data based on the estimated emotion.

11. The system according to claim 1, wherein the circuitry is further configured to adjust a level of detail of the plan data based on the goal of the user, such that the circuitry generates the plan data with a first level of detail when the goal is a first goal type, and generates the plan data with a second level of detail different from the first level of detail when the goal is a second goal type.

12. The system according to claim 1, wherein the circuitry is further configured to apply different generation algorithms according to the constraint of the user when generating the plan data, the different generation algorithms comprising allergen filtering and substitute item suggestion based on at least one of nutrient matching or similarity scoring.

13. The system according to claim 1, wherein the circuitry is further configured to estimate an emotion of the user and adjust a manner of expressing the list data based on the estimated emotion, such that when the estimated emotion indicates stress, the circuitry generates the list data in a simplified format, and when the estimated emotion indicates relaxation, the circuitry generates the list data in a detailed format.

14. The system according to claim 1, wherein the circuitry is further configured to calculate an importance score for each item in the list data based on at least one of a usage frequency, a storage duration, or a nutritional value, and adjust a level of detail of the list data for each item based on the importance score.

15. The system according to claim 1, wherein the circuitry is further configured to estimate an emotion of the user and adjust a manner of expressing the recommendation data based on the estimated emotion, such that when the estimated emotion indicates stress, the circuitry generates the recommendation data in a concise format, and when the estimated emotion indicates relaxation, the circuitry generates the recommendation data in a comprehensive format.

16. The system according to claim 1, wherein the circuitry is further configured to adjust an order of the recommendation data based on a relevance score computed from the preference of the user, such that recommendations matching the preference are ranked higher than recommendations not matching the preference.

17. The system according to claim 1, wherein the circuitry is further configured to analyze text data associated with a social media activity of the user to determine an action label, and adjust a type of the sensor data to be acquired from the client terminal based on the action label.

18. A system comprising:
a communication interface configured to communicate with a client terminal via a packet-switched network;
a processor;
a random access memory;
a memory storing a data generation model obtained by machine learning and an emotion identification model obtained by machine learning;
a database; and
circuitry configured to:
receive, from the client terminal via the communication interface, sensor data representing a physiological state of a user, the sensor data comprising at least one of heart rate data, blood pressure data, body weight data, or sleep pattern data;
preprocess the received sensor data by applying at least one of noise removal, normalization, or missing value imputation;
analyze the preprocessed sensor data by inputting the preprocessed sensor data into the data generation model to generate plan data based on the physiological state, a goal, a preference, and a constraint of the user, the plan data comprising item identifiers, quantities, and a usage schedule optimized to minimize surplus items;
generate list data by extracting the item identifiers and the quantities from the plan data, integrating duplicate items, and adjusting the list data based on an inventory status stored in the database;
estimate an emotion of the user by inputting multimodal input data into the emotion identification model;
generate recommendation data based on the list data and context data associated with the user by applying a multivariate scoring algorithm, and adjust a manner of expressing the recommendation data based on the estimated emotion; and
transmit the recommendation data to the client terminal via the communication interface and the packet-switched network.

19. The system according to claim 18, wherein the circuitry is further configured to estimate the emotion of the user based on the multimodal input data comprising at least one of voice data, facial image data, or text data received from the client terminal, and wherein the emotion identification model comprises at least one of a multimodal Transformer, a convolutional neural network, or a large language model.

20. A method performed by circuitry of a system, the method comprising:
receiving, via a communication interface coupled to a packet-switched network, sensor data transmitted from a client terminal, the sensor data representing a physiological state of a user captured by a sensor of the client terminal;
analyzing the received sensor data by inputting the sensor data into a data generation model obtained by machine learning to generate plan data based on the physiological state, a goal, a preference, and a constraint of the user;
generating list data by extracting item identifiers and quantities from the plan data; and
generating recommendation data based on the list data and context data associated with the user, and transmitting the recommendation data to the client terminal via the communication interface and the packet-switched network.
